(19) United States
(12) Patent Application Publication
(10) Pub. No.: US 2026/0158043 A1
(43) Pub. Date: Jun. 11, 2026

(54) STEROID AS A MODULATOR OF GABAA RECEPTOR

(71) Applicant: UMECRINE COGNITION AB, Solna (SE)

(72) Inventors: Torbjörn BÄCKSTRÖM, Umeå (SE); Gianna RAGAGNIN, Umeå (SE); Magnus LÖFGREN, Umeå (SE); Jessica SJÖSTEDT, Gideå (SE)

(21) Appl. No.: 18/707,731

(22) PCT Filed: Nov. 10, 2022
(86) PCT No.: PCT/EP2022/081519

§ 371 Date: May 6, 2024

(30) Foreign Application Priority Data
Nov. 10, 2021 (EP) 21207630.1

(51) Int. Cl. A61K 31/566; A61P 3/04

(52) U.S. Cl. CPC A61K 31/566; A61P 3/04

(57) ABSTRACT

The present disclosure concerns the use of 3α-ethynyl-3β-hydroxy-5α-androstan-17-one as medicament, in particular in treatment of a disease or disorder associated with an α3 subtype of the GABA_{A} receptor. The present disclosure is specifically concerned treatment of obesity, hyperphagia disorders, Prader-Willi's syndrome, polycystic ovarian syndrome, and/or diabetes. Said invention is also concerned with reducing or preventing overweight.

### TECHNICAL FIELD

The present disclosure concerns the use of 3α-ethynyl-3β-hydroxy-5α-androstan-17-one as a medicament, in particular in treatment of a disease or disorder associated with an α3 subtype of the GABA_{A} receptor. The present disclosure is specifically concerned treatment of obesity, hyperphagia disorder, Prader-Willi's syndrome, polycystic ovarian syndrome, and/or diabetes. Said invention is also concerned with reducing and/or preventing overweight.

### BACKGROUND

The World Health Organization (WHO) have estimated that today nearly 2 billion adults worldwide, aged 18 years and older, are overweight. Obesity and overweight pose a major risk for chronic diseases, including type 2 diabetes, cardiovascular disease, hypertension and stroke, and certain forms of cancer. While energy balance is key to maintaining a healthy weight, genes are important in determining a person's susceptibility to weight gain.

Gamma-aminobutyric acid (GABA) is the main inhibitory neurotransmitter in the central nervous system, and in the rest of the body acting on the GABA_{A} and GABA_{B} receptors. The GABA system plays an important role in the regulation of eating behavior. An abrupt reduction of food intake is seen after hampering GABAergic transmission. In animal studies, local application of GABA_{A}-receptor agonists in key areas of feeding regulation in the brain has been shown to induce hyperphagia disorder. Excessive food intake is a well-known effect of GABA_{A} receptor modulating steroids (GAMS). This has been shown in both animal and human studies.

The GABA_{A} receptors are of several subtypes, located in different areas of the brain and are related to different CNS disorders and symptoms. Some GABA_{A} receptors are localized within a synapse (intra-synaptic) while others are located outside a synapse (extra-synaptic). Some GABA_{A} receptor modulating steroids can in physiological concentrations open the extra-synaptic GABA_{A} receptor by themselves (tonic inhibition) but not the intra-synaptic receptors (phasic inhibition). These two types of effects are dependent on different mechanisms on the GABA_{A} receptor, and the effects depend in addition on the subunit composition of the receptor. In addition, GAMS can enhance the effect of GABA in both extra and intrasynaptic receptors. The receptor subtype α4,β,δ is an extra-synaptic subtype with both tonic and phasic effects when subjected to 3α-hydroxy steroids, such as 3α-hydroxy-5α/β-pregnan-20-one/ol or 3α-hydroxy-5α/β-androstan-17-one/ol. The α3 subtypes are known to regulate feeding, hunger, and satiety. In the brain, mainly the α3β3γ2 receptor subtype is expressed.

Positive GABA_{A} receptor modulating steroids (GAMS) are metabolites of the sex and stress hormones pregnanolone, progesterone, deoxycorticosterone, cortisone and cortisol, known as pregnanolones; as well as the metabolites of testosterone, androstanedione and dehydroepiandrosterone, known as androstanes. GAMS have been the subject of various studies, at least partially elucidating their role in the neurological signal system in mammals. These steroid metabolites induce CNS symptoms and disorders. They may share a 3α-hydroxy group, a 5α or 5β pregnane or androstane steroid body, or a double bond between carbon atoms 4 and 5 and a ketone or hydroxy group on position 17, 20 or 21. Examples of such steroids are 3α-hydroxy-5α/β-pregnan/δ4-pregnen-20-one/ol steroids or 3αhydroxy-5α/β-androstan/δ4-androsten-17-one/ol steroids, such as allopregnanolone, tetrahydrodeoxycorticosterone and androstanediol. Another example of a GAMS is tetrahydrodeoxycorticosterone (THDOC).

As the 3α-hydroxy-pregnane/androstane steroids are endogenously produced and are metabolites of steroid hormones essential for life, their production cannot easily be interrupted. It was established previously that 3α-hydroxy-5α/β steroids may cause CNS disorders through the three possible mechanisms of a) direct action, b) tolerance induction, and/or c) withdrawal effect. These steroids are produced in high amounts during several days to years in specific disorders such as obesity, hyperphagia disorder, Prader-Willi's syndrome, polycystic ovarian syndrome, diabetes, during acute and chronic stress, the luteal phase of the menstrual cycle and during pregnancy. They are also continuously produced within the brain in high amounts at certain disorders. Their production is locally regulated. In certain disorder the specific receptor subtype is downregulated or not expressed at all. In such situations, the body compensates by expressing another receptor subtype. The α4,β,δ receptor type is often then overexpressed. The α4,β,δ receptor subtype is very sensitive to GAMS.

U.S. Pat. Nos. 5,232,917, 5,925,630, 5,939,545, 6,143,736 and 6,277,838 disclose a number of 3α-hydroxy steroids and 3β steroids. WO 99/45931 and WO 03/059357 disclose antagonistic effects of steroids. WO 08/063128 discloses a number of steroids, such as 3α-ethynyl-3β-hydroxyl-5α-androstan-17-oxime.

Wang et al. 2000 (Acta Physiol Scand 169, 333-341) and Wang et al. 2002 (J Neurosci 22(9):3366-75) discloses antagonistic effects of 3β-hydroxy-5α-pregnan-20-one and other 3β-hydroxy 5α/P pregnane steroids.

WO 09/142594 discloses 3α-ethynyl-3β-hydroxy-5α-androstan-17-one and teaches that this steroid has no effect as a GAMSA or GAMS (see table 3 in WO 09/142594).

As obesity and obesity related disorders are a large heath problem in the worlds, there is a great need to provide ways of treatment, alleviation and/or prevention of the above mentioned disorders. It remains a challenge to suppress the effects on GAMS and obtain blockers thereof in order to reduce the excessive food intake in a mammal in need thereof. Specific blockers, in particular blocker of α3 subunit of GABA_{A} receptors, are therefore needed as therapy. In addition, it remains a challenge to find compounds that are physiologically safe and suitable for pharmaceutical use, and which additionally are applicable in physiologically acceptable doses with reasonable time intervals, for treatment of obesity, hyperphagia disorders and diseases related to obesity and overweight.

### DISCLOSURE OF THE INVENTION

It is an object of the present invention to reduce or at least partly overcome challenges in the prior art. It is an object of the present invention to provide means for treatment, alleviation and/or prevention of obesity, hyperphagia disorders and diseases or disorders associated with obesity or related to obesity. It is an object to provide means for reducing weight in a mammal by suppressing positive GABA_{A} receptor modulating steroids (GAMS). In particular, it is an object to provide compounds useful in therapies to treat said disorders as well as compounds useful in non-therapeutic (in other words cosmetic) applications. It is also an object of the present invention to provide non-therapeutic treatments (in other words cosmetic) of overweight and overeating.

The present inventors have surprisingly found that the steroid compound 3α-ethynyl-3β-hydroxy-5α-androstan-17-one provides an antagonistic effect on GAMS enhancement of the α3 subtypes of the GABA_{A}-receptor-chloride ionophore complex. Therefore, this compound blocks the negative effects of GAMS, i.e. induction of an hyperphagia disorder and/or obesity. The compound thereby acts as a GAMS antagonist (GAMSA). As such, the present inventors show that 3α-ethynyl-3β-hydroxy-5α-androstan-17-one can be used in the treatment of GAMS-related and/or steroid-induced disorders or diseases of the central nervous system (CNS). These and other objects are achieved in full, or at least in part, by aspects of the inventive concepts as disclosed herein.

In a first aspect, there is provided the compound 3α-ethynyl-3β-hydroxy-5α-androstan-17-one, or a pharmaceutically acceptable salt, hydrate, prodrug or solvate thereof, for use as a medicament. The skilled person will appreciate that said medicament can be used in prevention, alleviation and/or treatment of a disease.

In a second aspect, there is provided the compound 3α-ethynyl-3β-hydroxy-5α-androstan-17-one, or a pharmaceutically acceptable salt, hydrate, prodrug or solvate thereof, for use in prevention, alleviation and/or treatment of a disease or disorder associated with an α3 subtype of the GABA_{A} receptor. Non-limiting examples of such diseases or disorders may be steroid-related CNS disorders or diseases, diabetes, or autoimmune diseases. Such steroid-related CNS disorder may for example be obesity, hyperphagia disorders and diseases or disorders associated with obesity or related to obesity. It is an object to provide means for reducing weight in a mammal by suppressing positive GABA_{A} receptor modulating steroids (GAMS). In particular, it is an object to provide compounds useful in therapies to treat said disorders. Said disease or disorder may thus be a disease or disorder associated with an α3 subtype of the GABA_{A} receptor as discussed herein.

In one embodiment, there is provided the compound 3α-ethynyl-3β-hydroxy-5α-androstan-17-one, or a pharmaceutically acceptable salt, hydrate, prodrug or solvate thereof, for use in prevention, alleviation and/or treatment of a disease or disorder selected from the group consisting of steroid-related CNS disorders or diseases, diabetes or autoimmune diseases. Detailed examples of diseases and disorders are discussed below.

In particular, said disease or disorder may be selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder, diabetes and autoimmune disease.

As used herein, the term “treatment” is used in the context of therapeutic treatment and relates to the treatment, such as causative or symptomatic treatment, of a disease or disorder, the alleviation of symptoms thereof and/or prevention of said disease or disorder. For example, it is envisioned that the obesity may be treated, alleviated or prevented by said treatment. For example, obesity may be a symptom of Prader-Willi's syndrome and may as such be treated, alleviated or prevented by said treatment.

For the sake of clarity and avoidance of any doubt, as used herein in the context of therapeutic applications the terms “3α-ethynyl-3β-hydroxy-5α-androstan-17-one” and “compound” are used interchangeably and are to be interpreted as encompassing 3α-ethynyl-3β-hydroxy-5α-androstan-17-one and any pharmaceutically acceptable salt, hydrate, prodrug and/or solvate thereof.

TABLE 1A
Example list of receptor subtypes of the GABA_{A} receptor.

| Isoform | Synaptic/Extrasynaptic | Anatomical location |
|---|---|---|
| α1β3γ2S | Both | Widespread |
| α2β3γ2S | Both | Widespread |
| α3β3γ2S | Both | Reticular thalamic |
| | | nucleus |
| α4β3γ2S | Both | Thalamic relay cells |
| α5β3γ2S | Both | Hippocampal pyramidal |
| | | cells |
| α6β3γ2S | Both | Cerebellar granule cells |
| α1β2γ2S | Both | Widespread, most |
| | | abundant |
| α4β3δ | Extrasynaptic | Thalamic relay cells |
| α6β3δ | Extrasynaptic | Cerebellar granule cells |
| α1β2 | Extrasynaptic | Widespread |
| α1β3 | Extrasynaptic | Thalamus, hypothalamus |
| α1β2δ | Extrasynaptic | Hippocampus |
| α4β2δ | Extrasynaptic | Hippocampus |
| α3β3θ | Extrasynaptic | Hypothalamus |
| α3B3ε | Extrasynaptic | Hypothalamus |

GAMSAs with specificity to an α3-subtype GABA_{A} receptor were unknown up to date. As shown in Table 1A, there are three known GABA_{A} receptor α3 subtype, namely α3β3γ2, α3β3θ and α3β3ε. The present inventors have found that when acting on the GABA_{A} receptor α3 subtype, 3α-ethynyl-3β-hydroxyl-5α-androstan-17-one is a partial antagonist to GABA and a full antagonist to 3α-hydroxy-pregnan/androstan-steroids. Exposure to a 3α-hydroxy-pregnan/androstan-steroid increases the chloride flux through the human GABA_{A} receptor of any subtype but 3α-ethynyl-3β-hydroxy-5α-androstan-17-one inhibits the chloride flux through the human GABA_{A} receptor α3 subtype. The effect is induced by GABA or induced by a 3α-hydroxy steroid combined with GABA. This has been tested in recombinantly expressed human embryonic kidney cells (HEK-cells) expressing the GABA_{A} receptor α3β2γ2 subtype, see the appended Examples (Example 2). Thus, in one embodiment, said α3 subtype of the GABA_{A} receptor is α3β3γ2. The inhibitory effect on GABAs own effect is surprising since it was known to a person of skill in the art that similar compounds (having a 3β-hydroxy configuration) have no antagonistic effect on GABA's own ability to open the receptor for flux of chloride ions. In prior art, similar compounds have either had no effect or have enhanced the effect of GABA and thus increased the chloride flux through the receptor (see for example U.S. Pat. No. 5,925,630).

Thus, herein is provided 3α-ethynyl-3β-hydroxy-5α-androstan-17-one or a pharmaceutically acceptable salt, hydrate, prodrug or solvate thereof, for use according to this aspect of the invention.

The molecular structure of 3α-ethynyl-3β-hydroxy-5α-androstan-17-one is (Formula 1)

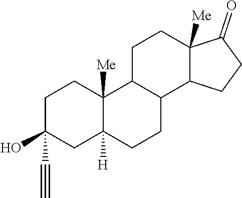 [SMILES: C#C[C@]1(O)CC[C@]2(C)C3CC[C@]4(C)C(=O)CCC4C3CC[C@H]2C1]

Interestingly, 3α-ethynyl-3β-hydroxy-5α-androstan-17-one efficiently antagonizes the GABA_{A} receptor modulation effect of 3α-hydroxy-5α/β-pregnan/androstan-steroids on the α3β3γ2 GABA_{A} receptor subtype. However, the compound has no effect, or a very small effect, in the more general GABA_{A} receptor subtype α1β2γ2 (Table 1B). As such, it is possible to selectively block the action of 3α-hydroxy-5α/β-pregnan/androstan-steroids on the α3β3γ2 GABA_{A} receptor by simultaneous administration of 3α-ethynyl-3β-hydroxy-5α-androstan-17-one in pharmaceutically and physiologically acceptable amounts. Thereby, it is plausible that major advantages are achieved when the compound of the present invention is administrated while elevated doses of GAMS (either endogenous or administered) or increased sensitivity to GAMS are present in the body or CNS of a subject. Without being bound by theory, it is envisioned that the selectively allows for administering a high dose when therapeutically motivated without the patient experiencing adverse side effects and/or allows for administering a low dose, for example during long term treatment, and still achieve a desired therapeutic outcome due to said selectivity.

Beneficially, this blocking may be achieved at pharmacologically and physiologically suitable concentrations as discussed in detail below.

According to one embodiment, there is provided 3α-ethynyl-3β-hydroxy-5α-androstan-17-one for use in antagonizing GABA signaling via the α3β3γ2 GABA_{A} receptor subtype.

According to the present disclosure, a GAMS is any steroid that positively modulates the GABA_{A} receptor. Typically, a positively modulating GAMS is a 3α-hydroxy-steroid. Non-limiting examples of such GAMS are 3α-hydroxy-5α/β-pregnan-20-one/ol, 3α-hydroxy-5α/β-androstan-17-one/ol and tetrahydrodeoxycorticosterone (THDOC, 3α, 21-dihydroxy-5α-pregnan-20-one).

Non-limiting examples of symptoms and conditions associated with or caused by the direct action of 3α-hydroxy-5α/β-steroids are obesity, hyperphagia disorder, Prader-Willi's syndrome, polycystic ovarian syndrome, diabetes, hepatic encephalopathy, sedation, tiredness, memory disturbance, learning disturbance, disturbance of motor function, clumsiness, increased appetite and food cravings, relapses in alcohol or substance abuse, negative mood as tension, irritability and depression which are the cardinal symptoms in the premenstrual syndrome and the worsening of Petit Mal epilepsy. The present inventors envision that the compound disclosed herein may be useful in the treatment of said diseases.

Thus, in one embodiment of the present disclosure, there is provided the compound for use a disclosed herein, wherein said disease or disorder associated with an α3 subtype of the GABA_{A} receptor is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance abuse substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; clumsiness, diabetes and autoimmune disease. The disease may also be obsessive-compulsive disorder.

The disease or disorder may be selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; obsessive-compulsive disorder and clumsiness, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness.

Also envisioned is that said compounds may be useful in treatment of fatty liver, insulin resistance, autoimmune disorders, and inflammatory disorders and symptoms. The present inventors envision that the compound disclosed herein may be useful in the treatment of said diseases or disorders.

Thus, in one embodiment, there is provided said compound for use as disclosed herein, wherein said disorder or disease selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration; ADHD; mobility disorders; essential tremor; Tourette's syndrome; balance disturbances; disturbance of motor function; and clumsiness, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome; menstrual cycle linked sleep disorders; attention disorders; menstrual cycle linked difficulties in concentration and ADHD; such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine; stress linked migraine; hypersomnia and in particular stress related hypersomnia; sedation; idiopathic hypersomnia; sleep disorders; fatigue syndrome; burn-out syndrome and menstrual cycle linked sleep disorders, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression; migraine; menstrual cycle linked migraine and stress linked migraine, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome; Alzheimer's disease; depression; stress related depression; premenstrual syndrome; premenstrual dysphoric disorder; menstrual cycle linked mood changes; negative mood such as tension, irritability and depression, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder; worsening of Petit Mal epilepsy; memory disturbance; learning disturbance; menstrual cycle linked memory changes; stress related memory changes; stress related learning difficulties; hepatic encephalopathy; Down's syndrome and Alzheimer's disease, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder; epilepsy; menstruation cycle dependent epilepsy; seizure disorder and worsening of Petit Mal epilepsy, such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder.

In one particular embodiment, said CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus. In one particular embodiment, said CNS disorder or disease is selected from the group consisting of alcoholism; substance use disorder and relapses into alcohol and/or substance use disorder.

In one embodiment of the present disclosure, there is provided the compound for use a disclosed herein, wherein steroid-related CNS disorder or disease is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; relapses into alcohol and/or substance use disorder, such as group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome and hyperphagia disorder associated with injury to the hypothalamus.

In one embodiment said disease or disorder is selected from hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus and diabetes; such as the group consisting of hyperphagia disorder, obesity, Prader-Willi's syndrome, polycystic ovarian syndrome, and diabetes.

In one embodiment, said disease or disorder is selected from the group consisting of obesity, hyperphagia disorder and Prader-Willi's syndrome; or the group consisting of obesity, hyperphagia disorder and polycystic ovarian syndrome; or the group consisting of obesity, hyperphagia disorder and diabetes. In one embodiment, said disease or disorder is selected from obesity and hyperphagia disorder.

Hyperphagia disorder relates to an abnormally great desire for food and/or excessive eating. Non-limiting examples of hyperphagia disorder comprise binge eating disorder, hyperphagia disorder associated with injury to the hypothalamus, and Prader-Willi's syndrome. A patient suffering from binge eating disorder suffers from recurrent episodes of eating large quantities of food and a feeling of loss of control. In one embodiment, said disease is a hyperphagia disorder, such as hyperphagia disorder resulting in overweight and/or obesity. In one embodiment, said hyperphagia disorder is binge eating disorder or hyperphagia disorder associated with injury to the hypothalamus. People suffering from Prader-Willi's syndrome have problems with hyperphagia disorder from young age and often become overweight or even obese already during the teenage years. These subjects typically exhibit an over expression of GABA_{A} receptor subunits that are highly sensitive to GAMS. Thus, in one embodiment, said disease or disorder is Prader-Willi's syndrome. In one embodiment, said disease or disorder is Prader-Willi's syndrome resulting in overweight and/or obesity. In other words, overweight and/or obesity associated with Prader-Willi's syndrome may be treated.

Obese children as well as obese adults have been reported to have high blood concentrations of GAMS. Thus, in one embodiment, said disorder or disease is obesity.

Said obesity may be hypothalamic obesity. Hypothalamic obesity refers to obesity that is caused by physical or inborn damage to the hypothalamus (Rose et al., 2018). The hypothalamus is part of the brain that makes hormones that control specific body functions such as sleep, body temperature, and hunger. It also makes hormones that control other organs in the body, especially the pituitary gland. The symptoms of hypothalamic obesity vary by the cause and include uncontrollable hunger, rapid, excessive weight gain, and a low metabolic rate. If the pituitary gland is involved, symptoms may include small, underdeveloped testes in males and delayed puberty. This condition most often occurs because of injury to the hypothalamus due to a tumor, swelling in the brain, brain surgery, or head trauma. The diagnosis is made by physical examination and review of the symptoms. There is no cure for hypothalamic obesity. At present, treatment involves a combination of surgery, medications, and nutritional and lifestyle counseling. The long-term outlook for people with this condition is dependent on weight loss and management.

For maintaining health, energy intake (in kilo calories) should normally be in balance with energy expenditure. For example, in the group of moderately active adults at age 66 or older, men are advised to eat about 2200 kilo calories per day and women are advised to eat about 1800 kilo calories per day. To avoid unhealthy weight gain, total fat should not exceed 30% of total energy intake. Intake of saturated fats should be less than 10% of total energy intake, and intake of trans-fats less than 1% of total energy intake, with a shift in fat consumption away from saturated fats and trans-fats to unsaturated fats, and towards the goal of eliminating industrially-produced trans-fats. In one embodiment, said hyperphagia disorder comprises eating at least 105% of the individual's energy expenditure, such as 110% of an individual's energy expenditure, such as 115% of an individual's energy expenditure, such as 120% of an individual's energy expenditure, such as 125% of an individual's energy expenditure, such as 130% of an individual's energy expenditure, such as 135% of an individual's energy expenditure, such as 140% of an individual's energy expenditure, such as 145% of an individual's energy expenditure, such as 150% of an individual's energy expenditure, such as 155% of an individual's energy expenditure, such as 160% of an individual's energy expenditure, such as 165% of an individual's energy expenditure, such as 170% of an individual's energy expenditure, such as 175% of an individual's energy expenditure, such as 180% of an individual's energy expenditure, such as 185% of an individual's energy expenditure, such as 190% of an individual's energy expenditure, such as 195% of an individual's energy expenditure, such as 200% of an individual's energy expenditure, such as 210% of an individual's energy expenditure, such as 220% of an individual's energy expenditure, such as 230% of an individual's energy expenditure, such as 240% of an individual's energy expenditure, such as 250% of an individual's energy expenditure, such as 260% of an individual's energy expenditure, such as 270% of an individual's energy expenditure, such as 280% of an individual's energy expenditure, such as 290% of an individual's energy expenditure, such as 300% of an individual's energy expenditure.

Women with PolyCystic Ovarian Syndrome (PCOS) have also been reported to exhibit high levels of GAMS. In one embodiment, said disease or disorder is polycystic ovarian syndrome. Over 60% of the women with this disorder are obese or overweight. Therefore, in one embodiment, said disease or disorder is polycystic ovarian syndrome, such as polycystic ovarian syndrome resulting in overweight and/or obesity. In another embodiment, said disease or disorder is obesity associated with to polycystic ovarian syndrome. In other words, overweight and/or obesity associated with polycystic ovarian syndrome may be treated.

Modulators of the GABA_{A} receptor can affect the insulin production, immunological functions and insulin resistance in diabetes type II (Tian et al., Prud'homme et al.) Thus, in a related embodiment, said disease or disorder is obesity associated with diabetes. In one related embodiment, said diabetes is diabetes type II.

Additionally, obesity and/or hyperphagia disorder may increase the risk for developing type II diabetes. Obesity and diabetes type II are a common comorbidity. In one embodiment, said diabetes is associated with overweight and/or obesity.

In recent years, a large body of scientific publication has shown that GABA signaling is also involved in the immune system and implicated disease of the immune system and in inflammation. It has become evident that cells of the immune system may also produce GABA and express GABA-A receptors. These extra synaptic channels can be activated by low nano to micromolar GABA concentrations, and such sub-micromolar GABA concentrations are present within the pancreas and in blood. The enzymes responsible for GABA synthesis and GABA-A receptors have been detected in all immunological competent cells e.g., T cells, macrophages, dendritic cells, macrophages monocytes and furthermore, GABAergic action is involved in the interactions between antigen presenting cells and T cells, between T and B cells in adaptive immune responses, or cytotoxic NK- and T-cell responses. The realization that extra synaptic GABA-A ion channels in immune cells can be fully activated by sub micromolar GABA concentrations makes GABA a potential effector molecule in many parts of the body including blood, pancreatic islets, cerebrospinal fluid and, of course, in the brain where the ambient GABA concentration is in the sub micromolar range. Research shows that mononuclear phagocytes and lymphocytes, dendritic cells, microglia, T cells and NK cells, express a GABAergic signaling machinery including membrane bound GABA-A receptors. Mounting evidence shows that GABA receptor signaling impacts important immune functions, such as cell migration, cytokine secretion, immune cell activation and cytotoxic responses (Bhandage, Barragan, 2021). Activation of GABA receptors on T cells and macrophages inhibits responses such as production of inflammatory cytokines. In T cells, GABA blocks the activation-induced calcium signal, and it also inhibits NF-κB activation. Furthermore, GABA clearly has an anti-inflammatory action, which is associated with inhibition of NF-κB activation. NF-κB activation is also blocked in pancreatic p cells, which may be of considerable therapeutic importance because this pathway induces apoptosis in these cells. (Prud'homme et al., 2015).

The absence of a presynaptic terminal defines these channels in the immune cells as extra synaptic-like channels existing in the brain. Physiologically this seems reasonable as the local concentration will be at nano or picomolar GABA concentrations close to immune cells in the blood when they enter the brain or the pancreatic islets. There are significant differences between the GABAergic stimulation depending on the GABA-A receptor subunit composition and therefore what subtypes are expressed in the immune cells. Properties like the affinity for GABA and the sensitivity to modulators' such as the benzodiazepines and steroids determine the effect of modulators (Lindquist and Birnir 2006; Olsen and Sieghart 2009). It is, therefore, of great importance if the modulators are specific to a certain subunit composition. GABA-A receptor subunit expression can be regulated with pharmacological agents (Uusi-Oukari, Korpi 2010). Positive GABA_{A} receptor modulating steroids (GAMS) are such agents that can change the effect of GABA at the GABA-A receptor. Another set of active compounds are positive GABA-A receptor steroid antagonists (GAMSA's) and particular 3α-ethynyl-3β-hydroxy-5α-androstan-17-one. In all situations where GABA acts via a GABA-A receptor, the GABA-A receptor modulating steroids can act and, in such situations, GAMSA can act as an antagonist on the particular receptor subtype. In the case that the GAMSA also has direct partial antagonistic effect against GABA then the GAMSA can have effect without presses of a GAMS. GAMSA can therefore have effect on the immune system via the GABAergic signaling.

Several studies have shown that GABA signaling may play a role in autoimmune disease and immune system related disease. For example, GABA ameliorates ongoing paralysis in experimental autoimmune encephalomyelitis (EAE) in mice models, by inhibiting onset of inflammation. GABA has also a role in rheumatoid arthritis and inflammatory responses to infection (Tian et al. 2011), autoimmune diseases like psoriasis, multiple sclerosis (Bath et al 2010), type I diabetes (Li et al 2017). Furthermore, the implication of GABA signaling in various autoimmune diseases, such as indicates a general role in inflammatory responses. In type 1 diabetes, GABA has been shown to have protective and stimulatory effects on β cells, but suppressive effects on the autoimmune response. (Prud'homme et al., 2015). Thus, in yet another embodiment, said disease or disorder is an autoimmune disease.

In one embodiment, said autoimmune disease may be diabetes type 1. Obesity in diabetes type I is a disadvantage since it may be harder to control the diabetes and maintain insulin at healthy levels. For example, if it is hard to control the intake of food it will be hard to control the dosing of insulin. This may lead to an elevated probability to reach a hypoglycemic state. The GABA-system is involved in diseases such as alcoholism and drug abuse. Modulators of the GABA_{A} receptor can affect the urge of abusing alcohol and/or substances. In this context, the substance may be any substance whose ingestion can result in a euphoric (“high”) feeling. In one embodiment, wherein said CNS disorder or disease is alcoholism, substance use disorder or relapse into alcoholism and/or substance abuse disorder. In one embodiment said disease or disorder is alcoholism. In one embodiment, said disease or disorder is substance use disorder.

As used herein the term, “drug use disorder” or “substance use disorder”, refers to a disease that affects a person's brain and behavior and leads to an inability to control the use of a legal or illegal drug or medication. Substances such as marijuana and nicotine also are considered drugs.

As used herein, the terms “alcoholism” and “alcohol use disorder” are used interchangeably.

The GABA system may be involved in the pathophysiology of obsessive-compulsive disorder. Thus, in one embodiment, said disease or disorder is obsessive-compulsive disorder.

It is envisioned that the administration of 3α-ethynyl-3β-hydroxy-5α-androstan-17-one or a pharmaceutically acceptable salt, hydrate, prodrug or solvate thereof leads to a decrease of bodyweight. For example, the decrease in bodyweight may be seen after 1 to 100 days, such as 2 to 20 days, such as after 3 to 15 days, such as after 5 to 10 days. Thus, in one embodiment said treatment results in a decrease in bodyweight after 1 to 100 days, such as 2 to 20 days, such as after 3 to 15 days, such as after 5 to 10 days, of treatment. In one particular embodiment, said treatment results in a decrease of daily calory intake by at least about 10%, such as at least 15%, such as at least 20%, such as at least 25%, such as at least 30%, such as at least 35%, such as at least 40%, such as at least 45%, such as at least 50%.

Naturally occurring steroids are subject to intense metabolism and are typically not suitable for oral administration as they quickly become degraded without sufficient time to exert its desired pharmacological effect. The present invention provides the use of a synthetic steroid with high water solubility compared to other steroids known to affect GABA signaling. In addition, the presence of an ethynyl moiety in position 3 (3α) has been shown to be able to prolong the half-life of a steroidal compound within the body of a mammal. The effect may be through preventing metabolic oxidations or degradation in said body.

3α-ethynyl-3β-hydroxy-5α-androstan-17-one may form salts which are within the scope of the present invention. Salts which are suitable for use in medicine are those wherein a counterion is pharmaceutically acceptable. The skilled person is aware of suitable salts for use in medicine. For example, suitable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)alkyl or aryl sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono, di- or tri lower alkylamine, for example ethyl, tertbutyl, diethyl, diisopropyl, triethyl, tributyl or dimethylpropylamine, or a mono-, di- or trihydroxy lower alkylamine, for example mono-, di- or triethanolamine. Corresponding internal salts may furthermore be formed.

In one embodiment, said pharmaceutically acceptable salt is a sodium salt. As apparent to a person of skill in the art, other salts may be equally suitable for the present compound. Non-limiting examples of other suitable salts are hydrochloride, sulfate, acetate, phosphate or diphosphate, chloride, potassium, maleate, calcium, citrate, mesylate, nitrate, tartrate and aluminum gluconate.

The compound 3α-ethynyl-3β-hydroxy-5α-androstan-17-one may exist as optical isomers and with deuterium or tritium instead of hydrogen; the invention encompasses compounds with all isotopes. In the synthesis of 3α-ethynyl-3β-hydroxy-5α-androstan-17-one, individual isomers may need to be separated by chromatographic techniques and/or by other separations methods.

According to the present disclosure, 3α-ethynyl-3β-hydroxy-5α-androstan-17-one may be administered by one of the following routes of administration: intravenously, nasally, per rectum, intravaginally, percutaneously, subcutaneously, transdermally, intramuscularly, or orally. In one embodiment, said 3α-ethynyl-3β-hydroxy-5α-androstan-17-one is administered by a route of administration selected from the group consisting of intravenous, nasal, per rectum, intravaginal, percutaneous, subcutaneous, transdermal, intramuscular and oral administration, such as the group consisting of nasal, per rectum, intravaginal, subcutaneous, transdermal, intramuscular and oral administration; such as the group consisting of nasal, subcutaneous, transdermal and oral administration. In one embodiment, said route of administration is selected from the group consisting of nasal, per rectum, intravaginal, percutaneous, subcutaneous, transdermal, intramuscular and oral administration, such as the group consisting of nasal, percutaneous, subcutaneous, transdermal and oral administration. In one embodiment, said route of administration is selected from the group consisting of nasal, oral and subcutaneous administration or nasal, oral and percutaneous administration. According to one embodiment, 3α-ethynyl-3β-hydroxy-5α-androstan-17-one is administered intravenously.

It is considered that ways of administration which are simple and easy for the patient, causing minimal discomfort if any, are desirable and also increase patient compliance with treatment. Nasal administration is envisioned as a promising administration alterative, as it offers the benefits of ease and the possibility of self-administration by a patient. Similarly, oral administration is envisioned as a promising administration alternative also allowing for self-administration without assistance of others. Thus, according to another embodiment, 3α-ethynyl-3β-hydroxy-5α-androstan-17-one is administered nasally. In one embodiment, 3α-ethynyl-3β-hydroxy-5α-androstan-17-one is administered orally.

Self-administration has the advantage of allowing a patient to adjust the dose or the frequency of medication either according to a subjective evaluation of their condition or according to a schedule prescribed by a treating physician. The term “schedule prescribed by the treating physician” includes the alternative where a patient makes a subjective evaluation of his/her condition, either unaided or aided by a questionnaire or a range or scale, or using an algorithm or a computer program, indicating the suitable next dose. Percutaneous administration, using 3α-ethynyl-3β-hydroxy-5α-androstan-17-one formulated as a cream, a gel, and an ointment or in the form of slow-release adhesive medicine patches, is another possible form of administration, similarly suitable for self-medication. The advantages of self-administration listed above apply also to percutaneous administration, with the added advantage that the administration can easily be interrupted if desired or necessary, e.g. by removing the medicine patch.

It is also possible that administration is via a depot formulation, which releases an effective amount of the therapeutically active compound as disclosed herein, over a period of time. The skilled person will appreciate that the depot formulation may be adapted to deliver the desired effective dose as prescribed by a treating physician. A depot formulation may be a subcutaneous depot formulation. Thus, in one embodiment, said administration via a depot formulation, such as a subcutaneous depot formulation.

In any of these or other routes of administration, the formulation of the composition may be adapted or adjusted according to normal pharmacological procedures, comprising the effective pharmaceutical in a chemical form, suitable for the chosen route, together with suitable excipients, such as adjuvants, carriers, diluents and vehicles, conventionally used and well-known to a person skilled in the art.

Conventionally used adjuvants and vehicles for oral administration are for example fillers or suspending agents like titanium dioxide, lactose anhydride, silica, silica colloidalis, methylcellulose, magnesium stearate, microcrystalline cellulose and the like. As used herein, the term “adjuvant” relates to a compound which potentiates the effect of the pharmaceutically active compound.

Conventionally used excipients for intravenous administration are for example sterile water for injections (WFI), sterile buffers (for example buffering the solution to pH 7.4) albumin solution, lipid solutions, cyclodextrin and variants thereof, and the like.

Conventionally used excipients for subcutaneous administration are for example sterile water for injections (WFI), sterile buffers (for example buffering the solution to pH 7.4) lipid solutions, cyclodextrins and the like. Conventionally used excipients for subcutaneous administration via a subcutaneous delivery system, such as a subcutaneous rod, are for example sterile water for injections (WFI), sterile buffers (for example buffering the solution to pH 7.4) lipid solutions, cyclodextrins and the like.

Conventionally used excipients for transdermal and/or subcutaneous administration are for example vaseline, liquid paraffin, glycerol, water, MCT oil, sesame oil and the like.

The skilled person will appreciate that the suitable dose will naturally vary depending on the mode of administration, the particular condition to be treated or the effect desired, gender, age, weight and health of the patient, as well as possibly other factors, evaluated by the treating physician. According to the present disclosure, 3α-ethynyl-3β-hydroxy-5α-androstan-17-one may be administered intravenously, a suitable dose may be that ranging from about 0.1 to about 300 mg per kg body weight. Preliminary studies in animals indicate that a preferred dose interval for intravenous administration is from about 20 to about 100 mg per kg body weight. In one embodiment, said compound is administrated in an effective dose in the range of from about 0.1 to about 300 mg per kg body weight, such as in a dose in the range of from about 0.2 to about 200 mg per kg body weight, such as in a dose in the range of from about 0.3 to about 150 mg, such as in a dose in the range of from about 0.4 to about 150 mg per kg bodyweight, such as in a dose in the range of from about 0.5 to about 120 mg per kg bodyweight, such as in a dose in the range of from about 1 to about 100 mg per kg body weight, such as in a dose in the range of from about 1 to about 50 mg per kg body weight, such as in a dose in the range of from about 1 to about 5 mg per kg body weight, such as about 1 mg per kg body weight.

It is envisioned that a therapeutically effective concentration of 3α-ethynyl-3β-hydroxy-5α-androstan-17-one may be in the range of from about 10 mg/day to about 30 g/day, such as of about 20 mg/day to about 20 g/day. In one embodiment, said compound is administrated in a dose in the range of from about 30 mg to about 15 g/day, such as in an effective dose in the range of from about 40 mg/day to about 15 g day, such as in a dose in the range of from about 50 mg/day to about 12 g/day, such as in a dose in the range of from about 100 mg/day to about 10 g/day, such as in a dose in the range of from about 100 mg/day to about 5 g/day, such as in a dose in the range of from about 100 mg/day to about 500 mg/day. In one embodiment, said compound is administrated in a dose in the range of from about 20 mg/day to about 60 g/day, such as in a dose in the range of from about 40 mg/day to about 40 g/day, such as in a dose in the range of from about 60 mg/day to about 30 g/day, such as in a dose in the range of from about 80 mg/day to about 30 g/day, such as in a dose in the range of from about 100 mg/day to about 24 g/day, such as in a dose in the range of from about 200 mg/day to about 20 g/day, such as in a dose in the range of from about 200 mg/day to about 10 g/day, such as in a dose in the range of from about 200 mg/day to about 1 g/day.

Said above mentioned doses are to be understood to refer to therapeutically effective doses.

As apparent to a person of skill in the art, 3α-ethynyl-3β-hydroxy-5α-androstan-17-one may be administered at one or more occasions per day. In one embodiment, 3α-ethynyl-3β-hydroxy-5α-androstan-17-one is administered once per day. In another embodiment, 3α-ethynyl-3β-hydroxy-5α-androstan-17-one is administered twice per day, or even three or four times per day. It may be suitable that the administration is in connection with meals, such as the three main meals of the day (for example breakfast, lunch and dinner or other meal schedule which is relevant and suitable for the patient, for example a more frequent meal schedule). It may also be suitable that the compound of the invention is administrated less frequently, such as every second day, or every third day, or even once every week. In embodiments where 3α-ethynyl-3β-hydroxy-5α-androstan-17-one is administrated as a subcutaneous implant or depot, the administration of the compound is a continuous process under diffusion. Such implant or depot may be inserted to a patient and may last for at least one month, such as for at least six months, such as for at least one year, such at least for two years, such as for at least three years, such as for at least four years, such as for at least five years, such as for at least six years. In one embodiment, wherein the compound is administrated as a subcutaneous implant or depot, it may be beneficial that the daily dose of is constant over a desired therapeutic period. The daily dose administered may be as discussed in the section above and is not repeated here for the sake of brevity.

In one embodiment, said compound for use as disclosed herein provides an antagonistic effect on the effect of γ-aminobutyric acid (GABA) and/or the effect of any GABA_{A} receptor modulating steroids (GAMS) on a GABA_{A} receptor α3 subtype, such as on the GABA_{A} receptor α3β2γ2 subtype. In one embodiment, said compound for use as disclosed herein provides an antagonistic effect on the effect of γ-aminobutyric acid (GABA) on said GABA_{A} receptor α3 subtype.

As described above, GABA is the main inhibitory neurotransmitter in the central nervous system, and in the rest of the body. Therefore, it is envisioned, without being bound by theory, that a 100% antagonistic effect of GABA may give rise to serious side effects. As such, said antagonistic effect achieved by the compound as disclosed herein is preferably a partial antagonistic effect. Thus, in one embodiment the antagonistic effect of said compound on GABA signaling via the α3 subtype GABA_{A} receptor is least 1%, such as at least 2%, such as at least 3%, such as at least 4%, such as at least 5%, such as at least 10%, such as at least 15%, such as at least 20%, such as at least 25%, such as at least 30%, such as at least 35%, such as at least 40%, such as at least 45%, such as at about 50%.

Preferably, said partial antagonistic effect is at most 80%, such as at most 75%, such as at most 70%, such as at most 65%, such as at most 60%, such as at most 55%, such as at most 50%.

Thus, in one embodiment said compound antagonizes GABA signaling via the α3 subtype GABA_{A} receptor by at most 80%, such as at most 75%, such as at most 70%, such as at most 65%, such as at most 60%, such as at most 55%, such as at most 50%.

For clarity, an antagonistic effect of 70% is to be interpreted as that 30% activity still remains.

The skilled person will appreciate that the compound for use as disclosed herein may be administered at a dose which achieves said partial antagonistic effect.

As discussed above, said compound provides an antagonistic effect on the effect of γ-aminobutyric acid (GABA) and/or any GABA_{A} receptor modulating steroids (GAMS) on an α3 subtype of the GABA_{A} receptor, such as the α3β2γ2 subtype of the GABA_{A} receptor. In one embodiment, said compound for use as disclosed herein further provides an antagonistic effect on the effect of γ-aminobutyric acid (GABA) and/or any GABA_{A} receptor modulating steroids (GAMS) on the GABA_{A} receptor α1, α2, α4 and/or α5 subtype, such as the α2, α4 and/or α5 subtype. In one embodiment, said compound further provides an antagonistic effect on the effect of γ-aminobutyric acid (GABA) and/or any GABA_{A} receptor modulating steroids (GAMS) on the GABA_{A} receptor α3 and α1 subtypes. In one embodiment, said compound further provides an antagonistic effect on the effect of γ-aminobutyric acid (GABA) and/or any GABA_{A} receptor modulating steroids (GAMS) on the GABA_{A} receptor α3 and α2 subtypes. In one embodiment, said compound further provides an antagonistic effect on the effect of γ-aminobutyric acid (GABA) and/or any GABA_{A} receptor modulating steroids (GAMS) on the GABA_{A} receptor α3 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α3 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α1 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α4 subtypes. In one embodiment, said compound further provides an antagonistic effect on the effect of γ-aminobutyric acid (GABA) and/or any GABA_{A} receptor modulating steroids (GAMS) on the GABA_{A}receptor α3, α1 and α5 subtypes. In one embodiment, said compound further provides an antagonistic effect on the effect of γ-aminobutyric acid (GABA) and/or any GABAA receptor modulating steroids (GAMS) on the GABA_{A} receptor α3, α2 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α2 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α3, α4 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α1, α2, α3 and α4 subtypes. In one embodiment, said compound further provides an antagonistic effect on the effect of γ-aminobutyric acid (GABA) and/or any GABA_{A} receptor modulating steroids (GAMS) on the GABA_{A} receptor α1, α2, α3 and α5 subtypes. In one embodiment, said compound further provides an antagonistic effect on the effect of γ-aminobutyric acid (GABA) and/or any GABA_{A} receptor modulating steroids (GAMS) on the GABA_{A} receptor α1, α3, α4 and α5 subtypes.

As discussed above, said compound provides an antagonistic effect on the effect of γ-aminobutyric acid (GABA) on an α3 subtype of the GABA_{A} receptor, such as the α3β2γ2 subtype of the GABA_{A} receptor. In one embodiment, said compound for use as disclosed herein further provides an antagonistic effect on the effect of γ-aminobutyric acid (GABA) on the GABA_{A} receptor α1, α2, α4 and/or α5 subtype, such as the α2, α4 and/or α5 subtype. In one embodiment, said compound further provides an antagonistic effect on the effect of γ-aminobutyric acid (GABA) on the GABA_{A} receptor α3 and α1 subtypes. In one embodiment, said compound further provides an antagonistic effect on the effect of γ-aminobutyric acid (GABA) on the GABA_{A} receptor α3 and α2 subtypes. In one embodiment, said compound further provides an antagonistic effect on the effect of γ-aminobutyric acid (GABA) on the GABA_{A} receptor α3 and α4 subtypes. In one embodiment, said compound further provides an antagonistic effect on the effect of γ-aminobutyric acid (GABA) on the GABA_{A} receptor α3 and α5 subtypes. In one embodiment, said compound further provides an antagonistic effect on the effect of γ-aminobutyric acid (GABA) on the GABA_{A} receptor α3, α2 and α1 subtypes. In one embodiment, said compound further provides an antagonistic effect on the effect of γ-aminobutyric acid (GABA) on the GABA_{A} receptor α3, α2 and α4 subtypes. In one embodiment, said compound further provides an antagonistic effect on the effect of γ-aminobutyric acid (GABA) on the GABA_{A} receptor α3, α1 and α5 subtypes. In one embodiment, said compound further provides an antagonistic effect on the effect of γ-aminobutyric acid (GABA) on the GABA_{A} receptor α3, α2 and α4 subtypes. In one embodiment, said compound further provides an antagonistic effect on the effect of γ-aminobutyric acid (GABA) on the GABA_{A} receptor α3, α2 and α5 subtypes. In one embodiment, said compound further provides an antagonistic effect on the effect of γ-aminobutyric acid (GABA) on the GABA_{A} receptor α3, α4 and α5 subtypes. In one embodiment, said compound further provides an antagonistic effect on the effect of γ-aminobutyric acid (GABA) on the GABA_{A} receptor α1, α2, α3 and α4 subtypes. In one embodiment, said compound further provides an antagonistic effect on the effect of γ-aminobutyric acid (GABA) on the GABA_{A} receptor α1, α2, α3 and α5 subtypes. In one embodiment, said compound further provides an antagonistic effect on the effect of γ-aminobutyric acid (GABA) on the GABA_{A} receptor α1, α3, α4 and α5 subtypes.

The level or degree of the antagonistic effect of said compound on GABA signaling via the α2, α4 and/or α5 subtype GABA_{A} receptor may differ from the same antagonistic effect via the α3 subtype of the GABA_{A} receptor, such as the α3β2γ2 subtype of the GABA_{A} receptor. Thus, in one embodiment the antagonistic effect of said compound on GABA signaling via the α2, α4 and/or α5 subtype GABA_{A} receptor is within the range of 1-30%, such as within the range of 2-25%, such as within the range of 3-22%, such as within the range of 4-20%, such as within the range of 5-15%, such as within the range of 7-13%, such as within the range of 8-10%. It will be understood that said ranges are equally relevant for any one of said further provided antagonistic effects on the effect of γ-aminobutyric acid (GABA) and/or any GABA_{A} receptor modulating steroids (GAMS) on individual or subsets of GABA_{A} receptor subtypes (see above).

The present inventors have shown that 3α-ethynyl-3β-hydroxy-5α-androstan-17-one provides unique effect on the different GABA_{A} receptor subtypes, compared to other similar known steroid ligands for this receptor. Example 4 describes the effect of 3α-ethynyl-3β-hydroxy-5α-androstan-17-one and of THDOC on the GABA-mediated current response at the α1β2γ2L, α2β3γ2S, α4β3δ and α5β3γ2L subtypes of the GABA_{A} receptor.

As a comparison, the effect of THDOC on said subtypes is shown. THDOC acts as an agonist where it significantly enhances the GABA-mediated current at α1β2γ2L, α4β3δ and α5β3γ2L GABA_{A} receptor subtypes (Table 8).

Surprisingly, 3α-ethynyl-3β-hydroxy-5α-androstan-17-one significantly reduce the GABA mediated current at α2β3γ2S, α4β3δ and α5β3γ2L GABA_{A} receptor subtypes, but has no effect at α1β2γ2L. Moreover, 3α-ethynyl-3β-hydroxy-5α-androstan-17-one has no own effect in absence of GABA at the tested GABA_{A} receptor subtypes (Table 7). Thereby, it has been shown that the compound for use according to the present invention is a selective inhibitor with a specific effect among the GABA_{A} receptors.

As described in detail is the experimental section 3α-ethynyl-3β-hydroxy-5α-androstan-17-one further provides an antagonistic effect on the effect of γ-aminobutyric acid (GABA) and/or any GABA_{A} receptor modulating steroids (GAMS) on the GABA_{A} receptor α2, α4 and/or α5 subtype.

It will be appreciated that the GAMS antagonist described herein may form part of a pharmaceutical composition. Thus, in a third aspect, there is provided a pharmaceutical composition comprising 3α-ethynyl-3β-hydroxy-5α-androstan-17-one for use as described herein, and at least one pharmaceutically acceptable excipient(s).

As used herein, the term “excipient” encompasses adjuvants, carriers, diluents, and vehicles. The skilled person appreciates that any adjuvants, carriers, diluents and vehicles mentioned in connection with the second aspect as disclosed herein are suitable in said pharmaceutical composition and it is withing the knowledge of the skilled person to make the appropriate choice thereof. The skilled person appreciates that the pharmaceutical composition may be adapted to be suitable for the selected administration route as well as desired administered dose. Relevant doses and administration routes are disclosed in connection to the second aspect above. Non-limiting examples of suitable carriers are cyclodextrin, sterile water for injections (WFI), sterile buffers (for example buffering the solution to pH 7.4) albumin solution, lipid solutions, cyclodextrin variants and the like.

According to a fourth aspect of the invention, there is provided use of 3α-ethynyl-3β-hydroxy-5α-androstan-17-one or of a pharmaceutically acceptable salt, hydrate, prodrug or solvate thereof, for the manufacture of a medicament for the prevention, alleviation and/or treatment of a disease or disorder associated with an α3 subtype of the GABA_{A} receptor; or for the prevention, alleviation and/or treatment of a disease or disorder selected from the group consisting of steroid-related CNS diseases or disorders, diabetes and autoimmune disease. In one embodiment, said use is of 3α-ethynyl-3β-hydroxy-5α-androstan-17-one or of a pharmaceutically acceptable salt, hydrate or solvate thereof. In this aspect of the invention, the corresponding embodiments of the second aspect, are applicable and are not repeated here merely for the sake of brevity.

In particular, said disease or disorder may be selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder; such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; and hyperphagia disorder associated with injury to the hypothalamus; or the group consisting of alcoholism; substance use disorder; relapses into alcohol and/or substance use disorder, diabetes and autoimmune disease.

In particular, the disease or disorder may be a disease or disorder selected from the group consisting of obesity, hyperphagia disorder, Prader-Willi's syndrome, polycystic ovarian syndrome, and diabetes. The disease or disorder may be Prader-Willi's syndrome. The disease or disorder may be polycystic ovarian syndrome, resulting in overweight or obesity. The disease or disorder may be diabetes, resulting in overweight or obesity. The disease or disorder may be obesity. The disease or disorder may be hyperphagia disorder.

The pharmaceutically acceptable salt may be a sodium salt. Other salts apparent to a person of skill in the art are also plausible as disclosed in connection with the second aspect.

In a related fifth aspect, there is provided a method of treatment, alleviation and/or prevention of a disease or disorder associated with an α3 subtype of the GABA_{A} receptor, comprising the step of administering a pharmaceutically effective amount of 3α-ethynyl-3β-hydroxy-5α-androstan-17-one or a pharmaceutically acceptable salt, hydrate, prodrug or solvate thereof as disclosed herein or a pharmaceutical composition as disclosed herein, to a patient in need thereof. In one embodiment said method comprises the step of administering a pharmaceutically effective amount of 3α-ethynyl-3β-hydroxy-5α-androstan-17-one or a pharmaceutically acceptable salt, hydrate or solvate thereof as disclosed herein or a pharmaceutical composition as disclosed herein.

Alternatively, there is provided a method of treatment, alleviation and/or prevention of a disease or disorder selected from the group consisting of steroid-related CNS diseases or disorders, diabetes and autoimmune disease, comprising the step of administering a pharmaceutically effective amount of 3α-ethynyl-3β-hydroxy-5α-androstan-17-one or a pharmaceutically acceptable salt, hydrate, prodrug or solvate thereof as disclosed herein or a pharmaceutical composition as disclosed herein, to a patient in need thereof. It will be appreciated that the embodiments disclosed in connection with the second aspect, including embodiments related to dose, frequency of administration and route of administration, are equally relevant for this fifth aspect as for aspects one, two and three and are not repeated here merely for the sake of brevity.

Said disease or disorder may be selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder; such as the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; and hyperphagia disorder associated with injury to the hypothalamus; or the group consisting of alcoholism; substance use disorder; relapses into alcohol and/or substance use disorder; diabetes and autoimmune disease.

In one embodiment of the fifth aspect, said disease is selected from the group consisting of obesity, a hyperphagia disorder, Prader-Willi's syndrome, polycystic ovarian syndrome, and diabetes. In one embodiment, said disease or disorder is selected from the group consisting of obesity, hyperphagia disorder and Prader-Willi's syndrome; or the group consisting of obesity, hyperphagia disorder and polycystic ovarian syndrome; or the group consisting of obesity, hyperphagia disorder and diabetes. In one embodiment, said disease or disorder is selected from obesity and hyperphagia disorder. In one embodiment, said disease or disorder is selected from obesity and diabetes. In one embodiment, said diabetes is diabetes type II. In one embodiment, said disease or disorder is a hyperphagia disorder, such as hyperphagia disorder resulting in overweight and/or obesity, In one embodiment, said hyperphagia disorder is binge eating disorder or hyperphagia disorder associated with injury to the hypothalamus. In one embodiment, said disease or disorder is Prader-Willi's syndrome. In one embodiment, said disease or disorder is Prader-Willi's syndrome resulting in overweight and/or obesity. In other words, overweight and/or obesity associated with Prader-Willi's syndrome may be treated. Thus, in one embodiment, said disease or disorder is obesity. In one embodiment, said disease or disorder is polycystic ovarian syndrome, such as polycystic ovarian syndrome resulting in overweight and/or obesity. In another embodiment, said disease or disorder is obesity associated with to polycystic ovarian syndrome. In other words, overweight and/or obesity associated with polycystic ovarian syndrome may be treated. In a related embodiment, said disease or disorder is obesity associated with diabetes. In one related embodiment, said diabetes is diabetes type II. Obesity and diabetes type II are a common comorbidity. In one embodiment, said diabetes is associated with overweight and/or obesity. In yet another embodiment, said disease or disorder is an autoimmune disease. In one embodiment, said autoimmune disease or disorder may be diabetes type I.

As explained above, the 3α-ethynyl-3β-hydroxy-5α-androstan-17-one may be in the form of a compound or a pharmaceutically acceptable salt thereof. In one embodiment of the fifth aspect, said compound is in the form of a sodium salt.

In one embodiment, the method of treatment, alleviation and/or prevention as disclosed herein results in a decrease of bodyweight. Plausibly, a decrease in bodyweight may be seen after 1 to 100 days, such as 2 to 20 days, such as after 3 to 15 days, such as after 5 to 10 days. In one embodiment, said method results in a decrease of daily calory intake by at least about 10%, such as at least 15%, such as at least 20%, such as at least 25%, such as at least 30%, such as at least 35%, such as at least 40%, such as at least 45%, such as at least 50%.

The present disclosure also relates to a pharmaceutical composition for use in a method according to the fifth aspect. Thus, there is provided a pharmaceutical composition, which comprises 3α-ethynyl-3β-hydroxy-5α-androstan-17-one or a pharmaceutically acceptable salt, prodrug, hydrate or solvate thereof, and a pharmaceutically acceptable carrier, excipient and or diluent as disclosed above.

The present disclosure further encompasses the use of 3α-ethynyl-3β-hydroxy-5α-androstan-17-one, for the manufacture of a pharmaceutical composition as disclosed herein.

The compound as disclosed herein may also be useful for cosmetic applications. Thus, in sixth aspect of the present disclosure, there is provided a use of 3α-ethynyl-3β-hydroxy-5α-androstan-17-one, or a cosmetically acceptable salt, hydrate, precursor or solvate thereof, for non-medical reduction and/or prevention of overweight in a subject. The compound can also be a precursor, which is transformed into 3α-ethynyl-3β-hydroxy-5α-androstan-17-one in the body of a subject, similarly to a prodrug. In one embodiment, there is provided a use of 3α-ethynyl-3β-hydroxy-5α-androstan-17-one, or a cosmetically acceptable salt, hydrate or solvate thereof, for non-medical reduction and/or prevention of overweight in a subject. Thus, said use in a non-therapeutic use and may also be referred to a cosmetic use. The skilled person appreciates that the terms non-medical, non-therapeutic and cosmetic are synonymous in this context and exclude medical uses which include treatment and/or preventions of pathological conditions. It is envisioned that prevention of overweight comprises reducing calory intake. For example, reduction of calory intake by at least about 10%, such as at least 15%, such as at least 20%, such as at least 25%, such as at least 30%, such as at least 35%, such as at least 40%, such as at least 45%, such as at least 50%.

To clarify, said non-medical use relates to reduction or prevention in subjects who have a BMI of less than 30. Thus, in one embodiment, said reduction and/or prevention of overweight is in a subject who has a BMI<30.

In one embodiment, said use relates to prevention of overweight in a subject who has a BMI below 25, and optionally who wishes to maintain a BMI in the range of from about 18.5 to 24.9.

In one embodiment, said use relates to reduction of overweight in a subject who has a BMI in the range of 25 to 29.9, and who wishes to reduce the BMI to the range of from about 18.5 to 24.9.

The skilled person will appreciate that said compound may be administrated at a dose as disclosed in connection with the second aspect is equally applicable and is not repeated here for the sake of brevity. Similarly, the administration occasions disclosed in connection with the second aspect are also applicable here.

The present inventors envision, a use of a composition comprising 3α-ethynyl-3β-hydroxy-5α-androstan-17-one, wherein said composition is a cosmetic composition. Thus, there is provided a use of a cosmetic composition comprising 3α-ethynyl-3β-hydroxy-5α-androstan-17-one and at least one cosmetically acceptable excipient. The skilled person will appreciate the excipients disclosed in connection with the third aspect, relating to the pharmaceutical composition, also are applicable to the cosmetic composition and are not repeated here for the sake of brevity. Thus, said excipient are considered cosmetically acceptable excipients. Additionally, the skilled person is aware of other suitable cosmetic acceptable excipients.

The skilled person will appreciate that said compound may be administrated at a dose as disclosed in connection with the third aspect is equally applicable and is not repeated here for the sake of brevity. For clarity, thus said doses are considered cosmetically effective doses. However, in particular, it is envisioned that 3α-ethynyl-3β-hydroxy-5α-androstan-17-one is administered by a route of administration selected from the group consisting of nasal, percutaneous, subcutaneous, transdermal, and oral administration may be suitable administration routes. In one embodiment, said route of administration selected from the group consisting of nasal, transdermal, and oral administration. In one embodiment, said route of administration is oral administration.

It is envisioned that said use will lead to the decrease in bodyweight which may be seen after 1 to 100 days, such as 2 to 20 days, such as after 3 to 15 days, such as after 5 to 10 days. Thus, in one embodiment use results in a decrease in bodyweight after 1 to 100 days, such as 2 to 20 days, such as after 3 to 15 days, such as after 5 to 10 days, of treatment. In one particular embodiment, said use results in a decrease of daily calory intake by at least about 10%, such as at least 15%, such as at least 20%, such as at least 25%, such as at least 30%, such as at least 35%, such as at least 40%, such as at least 45%, such as at least 50%.

In a related aspect, there is provided a cosmetic, non-therapeutic method of preventing or reducing overweight in a subject comprising administering a cosmetically effective amount of a compound selected from the group consisting of 3α-ethynyl-3β-hydroxy-5α-androstan-17-one, or a cosmetically acceptable salt, hydrate, precursor or solvate thereof.

In one embodiment, there is provided said cosmetic, non-therapeutic method, wherein said prevention or reduction of overweight is in a subject having a BMI<30. In one embodiment, there is provided said cosmetic, non-therapeutic method, wherein said overweight is defined as a BMI in the range of 25-29.9. In one embodiment of said cosmetic, non-therapeutic method, a decrease in bodyweight is seen after 1 to 20 days, such as after 3 to 15 days, such as after 5 to 10 days. The skilled person appreciates that embodiments disclosed in the context of the sixth aspect above are also applicable to the cosmetic, non-therapeutic method as disclosed herein.

In a seventh aspect, there is provided a cosmetic composition comprising a cosmetically effective amount of 3α-ethynyl-3β-hydroxy-5α-androstan-17-one as shown in Formula 1 or a cosmetically acceptable salt, hydrate, precursor or solvate thereof and at least one cosmetically acceptable excipient. In one embodiment, there is provided cosmetic composition comprising a cosmetically effective amount of 3α-ethynyl-3β-hydroxy-5α-androstan-17-one as shown in Formula 1 or a cosmetically acceptable salt, hydrate or solvate thereof and at least one cosmetically acceptable excipient.

It is envisioned that said composition may comprise both said compounds. Acceptable excipients are discussed in detail in the context of the third aspect and are not repeated there for the sake of brevity. The cosmetic composition may be formulated for a route of administration selected from the group consisting of nasal, percutaneous, subcutaneous, transdermal, and oral administration, in particular group consisting of nasal, transdermal, and oral administration as discussed above. In one embodiment, said cosmetic composition is formulated for oral or nasal administration.

For the sake of clarity and avoidance of any doubt, as used herein in the context of non-therapeutic (in other words cosmetic) uses the terms “3α-ethynyl-3β-hydroxy-5α-androstan-17-one”, “compound” and “compounds” are used interchangeably and are to be interpreted as encompassing 3α-ethynyl-3β-hydroxy-5α-androstan-17-one and any cosmetically acceptable salt, hydrate, precursor and/or solvate thereof.

It is noted that, as used in this specification and the appended claims, the singular forms “a”, “an”, and “the” also include plural referents unless the context clearly dictates otherwise.

The term “blocking” is meant to define an effect where in this case GABA or the 3α-hydroxy-5α/β-steroids are prevented from acting on the GABA-R receptor. It is to be understood that “blocking” is an entirely different effect than meant by “modulation” or “repression” or similar terms, which suggest that an action is still taking place, but to a lesser extent or at a slower rate.

The term “pharmaceutical composition” is used in its widest sense, encompassing all pharmaceutically applicable compositions containing at least one active substance and optional carriers, adjuvants, diluents, constituents etc. The term “pharmaceutical composition” also encompasses a composition comprising the active substance in the form of derivate or a pro-drug, such as pharmaceutically acceptable salts, sulphates and esters. The manufacture of pharmaceutical compositions for different routes of administration falls within the capabilities of a person skilled in galenic chemistry.

The skilled person appreciates that a precursor is a compound that participates in a chemical reaction that produces another compound; and that a prodrug is a compound that, after intake, is metabolized (i.e., participates in a chemical reaction) within the body into a pharmacologically active drug (i.e., another compound). The terms “prodrug” and/or “precursor” are used herein to describe a compound that participates in a chemical reaction to form 3α-ethynyl-3β-hydroxy-5α-androstan-17-one as shown in Formula 1. Typically, the chemical reaction takes place upon after administration, or after administration of 3α-ethynyl-3β-hydroxy-5α-androstan-17-one. The skilled person will appreciate that many different types of prodrugs to 3α-ethynyl-3β-hydroxy-androstan-17-one may be suitable. For example, a position of Formula 1 may be protected by a protection group. For example, the 3β-hydroxy group of Formula 1 may be protected, thus forming a precursor. An example of such prodrugs may be an ester of said compound formed at the 3 position. The term “precursor” is used herein to describe a compound that participates in a chemical reaction to form 3α-ethynyl-3β-hydroxy-5α-androstan-17-one as shown in Formula 1. Typically, the chemical reaction takes place upon after administration, or after administration of 3α-ethynyl-3β-hydroxy-5α-androstan-17-one. The skilled person will appreciate that many different types of precursors to 3α-ethynyl-3β-hydroxy-androstan-17-one may be suitable. For example, a position of Formula 1 may be protected by a protection group. For example, the 3β-hydroxy group may be protected, thus forming a precursor. An example of such precursor may be an ester of said compound formed at the 3 position.

The skilled person appreciates that the prodrug or precursor may for example be activated intracellularly (for example via metabolic enzymes) and/or extracellularly (for example in the milieu of gastrointestinal fluids, within the systemic circulation and/or other extracellular fluid compartments or near therapeutic target tissues/cells, relying on common enzymes such as esterases and phosphatases or target directed enzymes).

The term “cosmetic composition” is used in its widest sense, encompassing all cosmetically applicable compositions containing at least one active substance and optional carriers, adjuvants, diluents, constituents etc. The term “cosmetic composition” also encompasses a composition comprising the active substance in the form of derivate or a precursor form, such as cosmetically acceptable salts, sulphates and esters. The manufacture of cosmetical compositions for different routes of administration falls within the capabilities of a person skilled art.

The terms “administration” and “mode of administration” as well as “route of administration” are also used in their widest sense. The pharmaceutical composition and cosmetic composition of the present invention may be administered in a number of ways depending largely on whether a local, topical or systemic mode of administration is most appropriate for the condition be treated. These different modes of administration are for example topical (e.g., on the skin), local (including ophthalmic and to various mucous membranes, for example vaginal and rectal delivery), oral, parenteral or pulmonary, including the upper and lower airways. The preparation of such compositions and formulations is generally known to those skilled formulation arts and may be applied to the formulation of the composition of the present invention.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art, and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compound can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically or cosmetically acceptable inert carrier (where appropriate) such as ethanol, glycerol, water, and the like.

Compositions of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical or cosmetic compositions comprising the present compounds, or, particularly in the case of extended release, by the use of devices such as subcutaneous implants or osmotic pumps. The present compounds can also be administered liposomally.

Typical unit dosage compositions are those containing an effective dose, as hereinbefore recited, or an appropriate fraction thereof, of the active ingredient.

It should be understood that in addition to the ingredients particularly mentioned above, the compositions of this invention may include other agents conventional in the art having regard to the type of formulation in question, for example those suitable for oral administration may include flavoring agents.

With the term “antagonist” is meant a substance that hinders another substance, an agonist, to induce its effect. In this application the terms antagonist and blocker are used interchangeably.

The term “obesity” refers to a condition in a patient having a BMI ≥30. The BMI may be ≥35. The BMI may be ≥38. The BMI may be ≥40.

The term “overweight” refers to a condition in a subject having a BMI>25 but <30.

The term “Prader-Willi's syndrome” refers to a condition in a patient having at least one error in chromosome 15. It also refers to a condition in a patient that is of similar symptomatology. This similar symptomatology forms the diagnosis of Prader-Willi syndrome in a subject that does not have a visible at least one error in chromosome 15. The skilled person is aware of the genetic mutations underlying Prader-Willi's syndrome and the symptomatology that forms the diagnosis of said syndrome in cases where the patient does not have a visible error on chromosome 15.

The term “Poly cystic ovarian syndrome” refers to a condition in a patient fulfilling the so called “Rotterdam criteria” as established by the American Society for Reproductive Medicine (ASRM) and the European Society for Human Reproduction and Embryology (ESHRE) at a meeting in Rotterdam in 2003.

The term “hyperphagia disorder” refers to an abnormally increased appetite for consumption of food. Hyperphagia disorder may be associated with injury to the hypothalamus.

The term “binge eating disorder” refers to a subject who suffers from recurrent episodes of eating large quantities of food and a feeling of loss of control. The full criteria for diagnoses of the disease is given in Diagnostic and Statistical Manual of Mental Disorders (DSM; latest edition: DSM-5, publ. 2013) as established by the American Psychiatric Association.

With the phrase “GAMSA” is meant compounds that only antagonize the action of positive GABA_{A} receptor modulating steroids. When such compounds only antagonize or block the action of positive GABA_{A} receptor modulating steroids, they have a “GAMSA effect”. The abbreviation “GAMSA” stands for GABA_{A} receptor modulating steroid antagonist.

As used herein, the term “patient” refers to an individual who is exhibits or is at risk of exhibiting symptom(s) of a disorder relating to obesity and/or hyperphagia disorder.

As used herein, the terms “α3 subtype GABA_{A} receptor” and “GABA_{A} receptor α3 subtype” are used interchangeably.

As used herein, when the term “about” or “approximately” is used in relation to a numerical value, it is to be interpreted as a range of ±10%, such as ±9%, such as ±8%, such as 7%, such as ±6%, such as ±5%, such as ±4%, such as ±3%, such as ±2%, such as ±1%. For example, when the value is stated to be about 10, this means that the value is in fact in the range of from 9 to 11, such as in the range of from 9.9 to 10.9, such as in the range of from 9.8 to 10.8, such as in the range of from 9.7 to 10.7, such as in the range of from 9.6 to 10.6, such as in the range of from 9.5 to 10.5, such as in the range of from 9.4 to 10.4, such as in the range of from 9.3 to 10.3, such as in the range of from 9.2 to 10.2, such as in the range of from 9.1 to 10.1. The skilled person knows that numerical values relating to measurements are subject to measurement errors which place limits on their accuracy. For this reason, the general convention in the scientific and technical literature is applied: the last decimal place of a numerical value indicates its degree of accuracy. Where no other error margins are given, the maximum margin is ascertained by applying the rounding-off convention to the last decimal place e.g. for a measurement of 3.5 cm, the error margin is 3.45-3.54. When interpreting ranges of values in patent specifications, the skilled person proceeds on the same basis.

While the invention has been described with reference to various exemplary aspects and embodiments, it will be understood by those skilled in the art that various changes may be made, and equivalents may be substituted for elements thereof without departing from the scope of the invention. Therefore, it is intended that the invention is not limited to any particular embodiment contemplated, but that the invention will include all embodiments falling within the scope of the appended claims. The invention will be further illustrated by the following non-limiting Examples.

### BRIEF DESCRIPTION OF THE DRAWINGS

FIG. 1 shows the antagonistic effect of 3α-ethynyl-3β-hydroxy-5α-androstan-17-one against (μM on the x-axis) GABA in the human GABA_{A} receptor α3-β3-γ2 subtype.

FIG. 2 shows the weight increase in grams in rats treated with 3α-ethynyl, 3β-hydroxy-androstan-17-one for five days. Grey stables are the increase from arrival of the rat and white stables from the start of the treatment. It is shown that treated rats exhibited a lower increase in weight compared to the control group. FIG. 2A shows the increase from the start of treatment and FIG. 2B shows the increase from arrival.

FIG. 3 shows the difference in weight after 10 days of treatment of rats with two different doses of 3α-ethynyl-3β-hydroxy-5α-androstan-17-one, vehicle or estradiol+progesterone. The weight difference is normalized from the vehicle mean.

FIG. 4 shows the effect of 3α-ethynyl-3β-hydroxy-5α-androstan-17-one on the GABA-mediated current response at the α1β2γ2L, α2β3γ2S, α4β3δ and α5β3γ2L subunits of the GABA_{A} receptor.

### REFERENCES

Tian et al., 2019, J. Diabetes Res., doi: 5783545
Prud'homme et al., 2015, Autoimmun Rev., 11, 1048-56
Wang et al. 2000, Acta Physiol Scand., 169, 333-341
Wang et al. 2002, J Neurosci., 22(9):3366-75
Frye et al. 2000, Pharmacol Biochem Behav, 67, 587-596
Rose et al. 2018. Obesity. 26(11):1727-1732
Bhandage et al. 2021, Cell Mol Life Sci, 78, 5667-5679
Lindquist et al. 2006, J Neurochem, 97(5):1349-1356
Olsen et al. 2009, Neuropharmacology, 56(1):141-148
Uusi-Oukari M et al, 2010, Pharmacol Rev, 62(1):97-135
Tian J et al. 2011, Autoimmunity, 44:465-470
Bhat R et al. 2010, Proc Natl Acad Sci USA, 107(6):2580-2585
Li J et al, 2017, Cell 168(1-2):86-100.e115

### Example 1+2: Testing of GABA_{A} Receptor Effects of 3α-Ethynyl-3β-Hydroxy-5α-Androstan-17-One on Human α1β2γ2L GABA_{A} Receptor and Human α3β3γ2 GABA_{A} Receptor Subtype

Aim: To investigate the effect of 3α-ethynyl-3β-hydroxy-5α-androstan-17-one on the GABA_{A} receptor function both in absence and in presence of the GAMS tetrahydrodeoxycorticosterone (THDOC) by the Dynaflow™ system (Fluicell) on HEK-293 cells recombinantly expressing human α1β2γ2L GABA_{A} receptor or human α3β3γ2 GABA_{A} receptor subtype. In these tests the protocol was optimized to be similar to the physiological conditions in the synaptic cleft.

Preparation of cell lines: Cell lines permanently expressing a functional human GABA_{A} receptor was made as follows. The GABA_{A} receptor subunits α1β2γ2L or α3β3γ2 including introduced Kozac sequences just before the start codons were subcloned into mammalian expression vectors comprising Geneticin, Hygromycin B, and Zeocin resistance, respectively. A HEK-293 cell line stably expressing the three GABA_{A} receptor subunits (α1β2γ2L, α3β3γ2) separately for α1β2γ2L and α3β3γ2 subunit composition was produced by transfection of the subunits one at a time. The transfection was followed by selection with the appropriate antibiotics, cell separation with the use of subunit specific antibodies (β2 and γ2, Abcam, Upstate/Chemicon/Milipore, Sigma Aldrich), and production of single cell colonies. Produced cell lines were analysed with immunocytochemistry for the three GABA_{A} receptor subunits, followed by selection of a suitable cell line showing for the GABA_{A} receptor normal and good reactivity towards GABA and THDOC.

HEK-293 cells (obtained from Pasteur Institute, Paris, France), permanently transfected with the human 1β2γ2L or α3β3γ2 GABA_{A} receptor subtypes, were seeded at a density of 3×10⁴/25 cm² in a culture flask. The transfected cells were used for patch-clamp experiments 3 days after seeding. When using the cells for patch-clamp experiments the cells were washed twice with oxygen-bubbled EC-solution (see below). About 5 mL EC was then added and the cells were kept in the incubator for about 15 minutes. After 15 minutes the cells came loose from the bottom of the flask and were separated by carefully sucking a couple of times with a Pasteur pipette.

Dynaflow™ system with Resolve chips (Fluicell) was used for all patch-clamp experiments. The DF-16 Proll chips (Fluicell) used have non-sticky inserts for the wells. The channel width was 150 μm and the height 50 μm. The well volume was 280 μL. Run time at the flow rate of 26 μL/min is 180 min. The pump settings were as follow: Omnifix 2 mL syringe with inner diameter of 9.65 mm was used. The syringe pump flow rate for DF-16 Pro II chip was 26 μL/min.

Steroids and GABA: GABA (Sigma Aldrich) was dissolved in EC-solution by ultrasound for about 40 minutes to the concentration of 10 mM in room temperature. All steroids were dissolved to the concentration of 6 mM in DMSO. The DMSO concentration was 0.1% in all end-solutions, including the wash solution (EC) and the solution with GABA alone. End-solutions are the solutions added into the wells of the chip.

Electrophysioloqy: Patch electrodes were pulled from 1.5 mm O.D., 0.86 mm I.D. borosilicate capillary glass without filament. Typical electrodes had a resistance of 2-5 MΩ when filled with intracellular solutions. The intracellular solution consisted of (in mM): 140 Cs-gluconate, 3.0 NaCl, 1.2 MgCl₂, 1.0 EGTA, 10 HEPES. pH was adjusted to 7.2 with CsOH. The extracellular (EC) solution used during recordings contained (in mM): 137 NaCl, 5.0 KCl, 1.0 CaCl₂), 1.2 MgCl₂, 10 HEPES, 10 glucose. pH was adjusted with NaOH to 7.4. All chemicals in the intracellular and extracellular solutions were obtained from Sigma-Aldrich. After compensating for the liquid junction potential, a steady holding potential of −17 mV was used in all experiments. In physiological conditions the HEK-293 has a resting potential at −40 mV and a low concentration of chloride ions inside the cell. By using the holding potential of −17 mV and the intracellular solution with low chloride ion concentration the chloride ions flux into the cell when the receptors are activated. All experiments were performed at room temperature (21 to 23° C.). The following standard protocol was used for all experiments.

GABA applications: By using the Dynaflow™ equipment it is possible to study transfected HEK-293 during almost physiological conditions. The Dynaflow™ system allows application of solutions for as short as 40 ms up to minutes in time. Physiologically, in the synaptic cleft, GABA is released in mM range for about 2 ms. In these experiments, GABA±steroid was applied for 40 ms. It was found that in almost all cells, the first GABA application gave a smaller response than the second GABA application. There was no difference in response between the second and the third GABA application. Therefore, the first GABA application is always repeated twice, and the second response was used in the analysis.

Washout: GABA is quite solubility in water and easy to washout from the receptor. The washout time was set to 1 min after application with GABA solely. Steroids on the other hand were difficult to dissolve in water and also difficult to washout from the receptor. In the experiments, THDOC (Steraloids) was used as the GABA agonist. With 2 minutes washout time, 200 nM THDOC had been completely washed out as shown by neither an accumulative nor a desensitization effect.

Incubation: To see the effect of the steroids and to achieve stable results it was found out that the steroids (THDOC and/or 3α-ethynyl-3β-hydroxy-5α-androstan-17-one Umecrine AB) had to be incubated on the receptor before application of GABA. Different incubation times were studied to achieve the optimal time for attaining stable results and minimize the washout time. Incubation time of 20 seconds showed to be the optimal time for washout time of 2 minutes.

The optimized protocol: The optimized protocol was the following: 20 s. incubation of steroids (THDOC and/or 3α-ethynyl-3β-hydroxy-5α-androstan-17-one), 40 milliseconds GABA±steroids (THDOC and/or 3α-ethynyl-3β-hydroxy-5α-androstan-17-one) application, 2 min. washout. The first GABA application is repeated twice with a washout time of 1 minutes between the first and the second application. The compound 3α-ethynyl-3β-hydroxy-5α-androstan-17-one was in concentrations 3 nM-10 μM and GABA in concentration 100 μM.

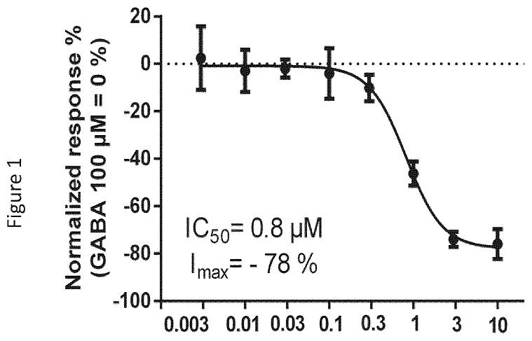

0.003-10 μM 3α-ethynyl-3β-hydroxy-5α-androstan-17-one was tested on 100 μM GABA-mediated current response at α3β3γ2L (FIG. 1). From the best fit curve the IC₅₀ was calculated to 0.8 μM and Iₘₐₓ was calculated to −78%. 0.3 μM 3α-ethynyl-3β-hydroxy-5α-androstan-17-one significantly reduce the GABA response with −10%. At lower concentrations there are some significance level, however, the effect is less than 10% and therefore considered as less important (Table 1B).

0.003-10 μM 3α-ethynyl-3β-hydroxy-5α-androstan-17-one was also tested in absence of GABA to study if the steroid activates the α3β3γ2L GABA_{A} receptor by itself. 3α-ethynyl-3β-hydroxy-5α-androstan-17-one do not activate the α3β3γ2L GABA_{A} receptor in absence of GABA (Table 1B).

TABLE 1B
The effect by 0.003-10 μM 3α-ethynyl-3β-hydroxy-5α-
androstan-17-one in presence and in absence of GABA at the α3β3γ2L
receptor subtype. Mean ± SEM % (N, P) EC=extracellular fluid, vehicle.

| | | 3α-ethynyl-3β-hydroxy- |
|---|---|---|
| 3α-ethynyl-3β-hydroxy- | 3α-ethynyl-3β-hydroxy- | 5α-androstan-17-one, |
| 5α-androstan-17-one | 5α-androstan-17-one + | Own effect, Baseline |
| conc. (μM) | 100 μM GABA | shift |
| 0.00/EC | — | +0.0 ± 0.2 pA |
| | | (N = 97) |
| 0.003 | +2.5 ± 2.9% | −1.0 ± 0.4 pA |
| | (N = 22, NS) | (N = 22) |
| 0.01 | −3.0 ± 2.3% | −0.7 ± 0.7 pA |
| | (N = 15, NS) | (N = 15) |
| 0.03 | −2.0 ± 1.0% | −0.8 ± 1.2 pA |
| | (N = 14, P = 0.000) | (N = 14) |
| 0.1 | −4.0 ± 3.0% | −1.0 ± 0.8 pA |
| | (N = 13, NS) | (N = 13) |
| 0.3 | −10 ± 2.3% | +0.9 ± 0.5 pA |
| | (N = 6, P = 0.000) | (N = 6) |
| 1 | −46 ± 1.3% | −1.2 ± 0.5 pA |
| | (N = 15, P = 0.000) | (N = 15) |
| 3 | −74 ± 0.9% | −0.4 ± 0.4 pA |
| | (N = 13, P = 0.000) | (N = 13) |
| 10 | −76 ± 1.9% | +1.1 + 2.4 pA |
| | (N = 11, P = 0.000) | (N = 11) |

Results and conclusion from Example 1: FIG. 1 shows the results of testing the antagonistic effect of 3α-ethynyl-3β-hydroxy-5α-androstan-17-one against GABA in receptor pharmacological studies on the human GABA_{A} receptor α3β3γ2 subtype. FIG. 1 demonstrations that 3α-ethynyl-3β-hydroxy-5α-androstan-17-one have an antagonistic effect against GABA's effect on the GABA_{A} receptor α3β3γ2 subtype (a total inhibitory effect of GAMS stimulated GABA effect).

Results Example 2: Table 2 shows the results of Example 2.

TABLE 2
Effect of 1 μM 3α-ethynyl-3β-hydroxy-5α-androstan-17-one on
receptor subype 1β2γ2 and α3β3γ2.

| Steroids present | α1β2γ2 | α3β3γ2 | Result |
|---|---|---|---|
| 3α-ethynyl-3β- | −1.9 ± 2.4% | −46 ± 1.3%, | Effect against GABAs |
| hydroxy-5α- | (NS; N = 22) | (P = 0.001; | agonistic effect |
| androstan-17-one | | N = 15) | |
| 1 μM | | | |
| 3α-ethynyl-3β- | No effect | −166 ± 19.9%, | Antagonism of 100 nM |
| hydroxy-5α- | | (P = 0.012; N = 8) | THDOC-enhancement |
| androstan-17-one | | | |
| 1 μM + THDOC | | | |

α1β2γ2 GABA_{A} receptors: 3α-ethynyl-3β-hydroxy-5α-androstan-17-one had no significant agonistic or antagonistic effect on GABA's effect alone or the combined effect of GAMS+GABA.

α3β3γ2 GABA_{A} receptors: 3α-ethynyl-3β-hydroxy-5α-androstan-17-one had a major antagonistic effect on the combined effect of THDOC+GABA (−166%) but had also an antagonistic effect on GABA alone however to a smaller degree (−46%).

Conclusion from Example 2: The results that are shown in Table 2 above show that 3α-ethynyl-3β-hydroxy-5α-androstan-17-one significantly antagonizes GABA in the α3β3γ2 GABA_{A} receptor subtype but has no effect in the more general subtype α1β2γ2.

Thus, the compound of the invention is very specific towards the α3β3γ2 receptor subtype.

### Example 3: Effect of 3α-ethynyl-3β-hydroxy-5α-androstan-17-one on Weight Increase in Animal Model of Growing Male Rats

### Treatment and Testing Schedule

Animals: Male Wistar rats were kept in group cages, with three animals per cage, from delivery and throughout the period of experiments. The individual animals were marked so that they could be identified throughout the experiment duration. The animals weight an average of 152 g upon arrival. A reversed light dark (12:12 h) cycle was used with the dark period onset at 0600 hrs. Altogether fifty-six (56) rats were used in this study (experiment 1+2). The animals were delivered from the breeder Taconic (Denmark). The study protocol was approved by the Regional Ethics Committee of Umea University, Sweden.

Feeding: Standard chow and water were available ad libitum. A reversed 12-h dark-light cycle with lights off at 10.00 am and lights on at 22.00 pm was used. For identification, the rats were marked with a permanent marker on the tail. To avoid the endogenous allopregnanolone fluctuations that are present in the oestrous cycle of female rats, male rats were chosen (Frye et al. 2000). The animals were allowed to acclimatize for at least 3 weeks before start of the experimental sessions. During this period, the rats were repeatedly handled and allowed habituation to the new environment and to all new procedures, to minimize stress during the experiments.

### Experimental Design

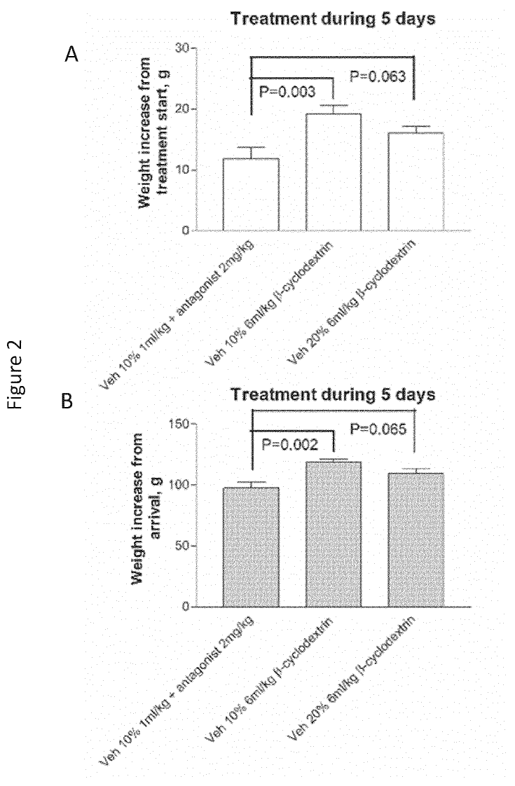

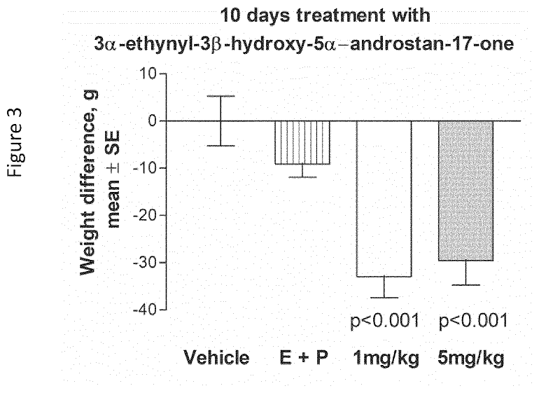

After 14 days of triad housing, handling and injection training of the animals, the animals were treated for 5 days with IV injections in experiment 1 and 10 days with a daily subcutaneous injection of the assigned treatment in experiment 2 (Table 3 and 4). The injections were given at 08.00 every morning. The animals were weighed the day before onset of the treatment and at the last day of injection i.e. on the 5th or 10th day of treatment. Weight was taken at the time of the last injection. A one-way ANOVA and non-parametric Kruscal-Wallice test showed differences for the treatment dosages compared to vehicle treatment. The results are shown in FIG. 2 (experiment 1) and FIG. 3 (experiment 2), respectively. Post hoc p-values are indicated in the Figures.

### Treatment Groups

TABLE 3
Treatment groups in experiment 1.

| | | |
|---|---|---|
| 3α-ethynyl-3β- | n = 7 | 3α-ethynyl-3β-hydroxy-5α-androstan-17-one, 2 mg/kg, |
| hydroxy-5α- | | dissolved in 10% 2-Hydroxypropyl-β-cyclodextrin |
| androstan-17- | | (Sigma-Aldrich), to 2 mg/ml, given was 1 ml/kg. |
| one | | |
| Vehicle 1 | n = 7 | 10% 2-Hydroxypropyl-β-cyclodextrin, 6 ml/kg |
| Vehicle 2 | n = 7 | 20% 2-Hydroxypropyl-β-cyclodextrin, 6 ml/kg |

TABLE 4
Treatment groups in experiment 2.

| | | |
|---|---|---|
| Vehicle | n = 8 | s.c. vehicle (sesame oil, Sigma-Aldrich) |
| E + P Control | n = 9 | s.c. 5 mg/kg progesterone (P) + 10 μg/kg 17β-estradiol |
| | | (E) s.c. Both Sigma-Aldrich |
| 3α-ethynyl-3β- | n = 9 | s.c. 3α-ethynyl-3β-hydroxy-5α-androstan-17-one; 1 mg/kg + |
| hydroxy-5α- | | 5 mg/kg progesterone + 10 μg/kg 17β-estradiol s.c. |
| androstan-17- | | |
| one low | | |
| 3α-ethynyl-3β- | n = 9 | s.c. 3α-ethynyl-3β-hydroxy-5α-androstan-17-one; 5 mg/kg + |
| hydroxy-5α- | | 5 mg/kg progesterone + 10 μg/kg 17β-estradiol |
| androstan-17- | | |
| one high | | |

### Results Example 3

Experiment 1: As can be seen in FIG. 2, there was no significant difference between the two vehicle treatments 10% or 20% 2-Hydroxypropyl-β-cyclodextrin. The increase in weight was significantly lower in the 3α-ethynyl-3β-hydroxy-5α-androstan-17-one-treated group than in the vehicle treated groups F(2,18)=6,293; p<0.008 when compared to weight at arrival to department or F(2,18)=6,103; p<0.009, or compared to weight at the start of treatment (Table 5). Ad hoc analysis of the weight increase from the arrival to the department shows a significant difference between 3α-ethynyl-3β-hydroxy-5α-androstan-17-one treatment and treatment with vehicle 10% (p<0.002) and a trend for significance against treatment with 20% β-cyclodextrin solution (p=0.065). The ad hoc test of the weight increase between start and end of treatment showed significant difference between the 3α-ethynyl-3β-hydroxy-5α-androstan-17-one treatment and vehicle treatment (p<0.003 for 10%) and a trend (p=0.063 for 20%). There was no significant difference between the weight changes in the vehicle treated groups. The cyclodextrin content in the 3α-ethynyl-3β-hydroxy-5α-androstan-17-one solution was 1 ml/kg while the cyclodextrin concentration in the vehicles was six times higher.

TABLE 5
Weight changes (mean, SEM gram) during vehicle and 3α-ethynyl-
3β-hydroxy-5α-androstan-17-one treatment

| | | Mean weight | |
|---|---|---|---|
| | | increase since | |
| Treatment | N | arrival, g | SEM |
| 3α-ethynyl-3β-hydroxy-5α-androstan- | 7 | 97.43 | 4.96 |
| 17-one, 2 mg/kg, 1 ml/kg, β- | | | |
| cyclodextrine, 10% | | | |
| Vehicle 10% 6 ml/kg β-cyclodextrine | 7 | 118.57 | 2.78 |
| Vehicle 20% 6 ml/kg β-cyclodextrine | 7 | 109.14 | 4.60 |
| | | Mean weight | |
| | | increase since | |
| | | treatment start, | |
| | N | g. | SEM |
| 3α-ethynyl-3β-hydroxy-5α-androstan- | 7 | 11.86 | 1.83 |
| 17-one, 2 mg/kg, 1 ml/kg, β- | | | |
| cyclodextrine, 10% | | | |
| Vehicle 10% 6 ml/kg β-cyclodextrine | 7 | 19.14 | 1.39 |
| Vehicle 20% 6 ml/kg β-cyclodextrine | 7 | 16.00 | 1.13 |

FIG. 2 shows the mean±SEM weight changes in grams (g) the weight difference between the weight at the start of the treatment minus the weight at five days later at the end of treatment (top, white stables) between the weight at arrival to the department minus the weight at the last day of treatment (bottom, grey stables). Antagonist 2 mg/kg=3α-ethynyl-3β-hydroxy-5α-androstan-17-one 2 mg/kg.

Experiment 2: The results show of 3α-ethynyl-3β-hydroxy-5α-androstan-17-one reduced the weight increase compared to controls treated with vehicle (placebo) or 5 mg/kg progesterone (P)+10 μg/kg 17β-estradiol (E) sub cutaneous (s.c.). The reduction in weight was surprising large as the weight already after 10 days of treatment differed over 20% compared to vehicle, see FIG. 3. The results indicate that 3α-ethynyl-3β-hydroxy-5α-androstan-17-one is very suitable to use as a treatment in e.g. adolescents with Prader-Willis syndrome.

Results and conclusion Example 3: FIG. 3 shows the weight difference (Mean±SE) normalized from the vehicle and estradiol (E)+progesterone (P) control mean (set as 0±SE g weight) in the groups treated with two dosages of 3α-ethynyl-3β-hydroxy-5α-androstan-17-one. The results show that 3α-ethynyl-3β-hydroxy-5α-androstan-17-one reduced the weight increase compared to controls treated with vehicle (placebo). Both dose groups, 1 mg/kg (n=9) and 5 mg/kg (n=9) showed a significant reduction in weight compared to vehicle (−33±4.4 g; and −29.6±5.2 g resp. p<0.001 in both groups, FIG. 3). The 3α-ethynyl-3β-hydroxy-5α-androstan-17-one treated groups showed also a significantly reduced weight versus the E+P control rats (−23.9±4.4 g vs. −20.4±5.2 g p<0.001 and p=0.003, FIG. 3). The reduction in weight was surprisingly large as the weight already after 10 days of treatment differed over 20%. The results indicate that 3α-ethynyl-3β-hydroxy-5α-androstan-17-one is very suitable to use in prevention, alleviation and/or treatment of a disease or disorder associated with an α3 subtype of the GABA_{A} receptor, such as obesity, hyperphagia disorder, Prader-Willi's syndrome, polycystic ovarian syndrome, and/or diabetes or for the non-therapeutic prevention, alleviation and/or treatment of overweight. The present inventors consider that the compound of the invention to be particularly suitable for use in a patient with Prader-Willis syndrome, such as in adolescents with Prader-Willis syndrome.

### Example 4: Receptor Pharmacological Studies of 3α-ethynyl-3β-hydroxy-5α-androstan-17-one and THDOC at α1β2γ2L, α2β3γ2S, α4β3δ and α5β3γ2L GABA_{A} receptor subtypes

Aim: the aim of this project was to study the effect of 3α-ethynyl-3β-hydroxy-5α-androstan-17-one and the positive GABA_{A} receptor modulator THDOC on GABA-mediated current response, at human α1β2γ2L, α2β3γ2S, α4β3δ and α5β3γ2L subunit GABAA receptors.

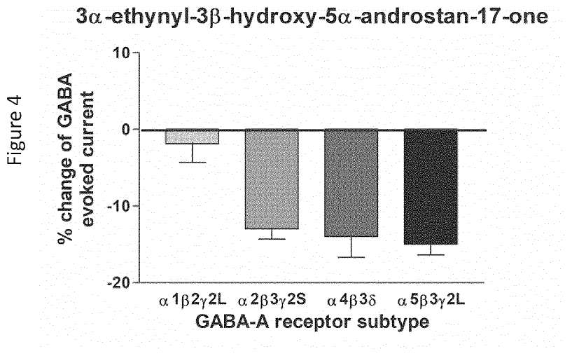

Study design: The current response mediated by chloride ion flux through the GABA_{A} receptors expressing the α1β2γ2L, α2β3γ2S, α3β3γ2L, α4β3δ and α5β3γ2L subunit was studied with the use of patch clamp technique combined with the Dynaflow™ application system and the Resolve chip. This provides rapid applications of and removal of substances. The material, protocol and methods were identical to the ones used in Examples 1 and 2. 1 μM 3α-ethynyl-3β-hydroxy-5α-androstan-17-one was studied on GABA mediated current response at α1β2γ2L, α2β3γ2S, α4β3δ and α5β3γ2L GABA_{A} receptor subtypes (FIG. 4, Table 6). In addition, the effect of 1-1000 nM THDOC was studied on GABA mediated current response at the α1β2γ2L, α3β2γ2, and α5β3γ2L receptor subtypes (Table 8). The binding site of THDOC is in the intracellular region of the receptor. To achieve stable response, the cells were exposed to steroid (THDOC) in 20 seconds before GABA application.

### Results

3α-ethynyl-3β-hydroxy-5α-androstan-17-one: 1 μM 3α-ethynyl-3β-hydroxy-5α-androstan-17-one significantly reduced the GABA mediated current at α2β3γ2S, α4β3δ and α5β3γ2L GABA_{A} receptor subtypes, without any effect at α1β2γ2L. The amount GABA used depends on the receptor subtype intra or extra-synaptic type, α1β2γ2L=30 μM; α2β3γ2S=45 μM; α4β3δ=1 μM and α5β3γ2L=0.3 μM GABA.

TABLE 6
The effect by 1 μM 3α-ethynyl-3β-hydroxy-5α-androstan-
17-one on GABA evoked current at the α1β2γ2L, α2β3γ2S,
α4β3δ and α5β3γ2L GABA_{A} receptor subtypes. Mean ± SEM % (N, P)

| | 3α-ethynyl-3β- |
|---|---|
| GABA_{A} receptor | hydroxy-5α- |
| subunit | androstan-17-one |
| α1β2γ2L | −1.9 ± 2.4% |
| | (N = 22, NS) |
| α2β3γ2S | −13 ± 1.3% |
| | (N = 11, P = 0.003) |
| α4β3δ | −14 ± 2.7% |
| | (N = 10, P = 0.005) |
| α5β3γ2L | −15 ± 1.4% |
| | (N = 26, P = 0.000) |

As shown in Table 7, 1 μM 3α-ethynyl-3β-hydroxy-5α-androstan-17-one has no effect in absence of GABA at the tested GABA_{A} receptor subtypes.

TABLE 7
The effect by 1 μM 3α-ethynyl-3β-hydroxy-5α-androstan-
17-one own effect on direct activation, in absence of GABA,
at α1β2γ2L, α2β3γ2S, α4β3δ and α5β3γ2L
GABAA receptor subtypes. Significance is calculated
when baseline shift ≥ 5 pA. Mean ± SEM pA (N, P
when baseline shift ≥ 5 pA), EC = vehicle = 0 μM.

| | | 3α-ethynyl-3β- |
|---|---|---|
| GABA_{A} | | hydroxy-5α- |
| receptor | | androstan-17- |
| subunit | EC | one |
| α1β2γ2L | +0.7 ± 0.2 pA | −0.1 ± 0.8 pA |
| | (N = 46) | (N = 22) |
| α2β3γ2S | −0.2 ± 0.6 pA | −7.5 ± 2.6 pA |
| | (N = 34) | (N = 11, P = |
| | | 0.002) |
| α4β3δ | +0.2 ± 0.4 pA | −0.8 ± 1.0 pA |
| | (N = 27) | (N = 11) |
| α5β3γ2L | −0.9 ± 0.4 pA | −3.9 ± 1.0 pA |
| | (N = 53) | (N = 20) |

THDOC: The following standard protocol was used for the different receptor subtypes:
α1β2γ2L: 20 s preincubation of 30-1000 nM THDOC 40 ms application of 30 μM GABA±steroids.
α3β2γ2: 20 s preincubation of 1-1000 nM THDOC followed by 1 s application of 100 μM GABA±steroids.
α5β3γ2L: 20 s preincubation of 30-1000 nM THDOC followed by 6 s application of 0.3 μM GABA±steroids.

TABLE 8
EC50 values and Emax values for the effect of THDOC on GABA
mediated current response of different receptor subtypes.

| Receptor subtype | EC₅₀ (nM) | Eₘₐₓ (%) |
|---|---|---|
| α1β2γ2L | 70 | 115 |
| α3β2γ2 | 45 | 80 |
| α5β3γ2L | 68 | 131 |

### Conclusion Example 4

As can be seen in FIG. 4, 3α-ethynyl-3β-hydroxy-5α-androstan-17-one does not have an effect on the α1β2γ2L receptor subtype but has a direct activation effect on the other three receptor subtypes. As can be seen in FIG. 1, and as discussed in Example 1, high concentrations (1-10 μM) of 3α-ethynyl-3β-hydroxy-5α-androstan-17-one reduce the GABA-mediated current at α3β3γ2L.

3α-ethynyl-3β-hydroxy-5α-androstan-17-one has no effect in absence of GABA.

3α-ethynyl-3β-hydroxy-5α-androstan-17-one, at higher concentrations of 1-10 μM, strongly reduces the GABA mediated current response at α3β3γ2 GABA_{A} receptor subunit (FIG. 1). 1 μM 3α-ethynyl-3β-hydroxy-5α-androstan-17-one had minor reducing effect on GABA mediated current response, approximately −15%, at α2β3γ2S, α4β3δ and α5β3γ2L GABA_{A} receptor subtypes but no effect on α1β2γ2L. 3α-ethynyl-3β-hydroxy-5α-androstan-17-one cannot activate the GABA_{A} receptor in absence of GABA.

To conclude, 3α-ethynyl-3β-hydroxy-5α-androstan-17-one is an antagonist on a subset of GABA_{A} receptor subtypes.

Furthermore, the effect of THDOC on different subtypes is shown. While 3α-ethynyl-3β-hydroxy-5α-androstan-17-one acts as an antagonist, THDOC acts as an agonist where it significantly enhances the GABA-mediated current at α1β2γ2L, α4β3δ and α5β3γ2L GABA_{A} receptor subtypes (Table 8).

### Itemized List of Embodiments

1. 3α-ethynyl-3β-hydroxy-5α-androstan-17-one as shown in Formula 1

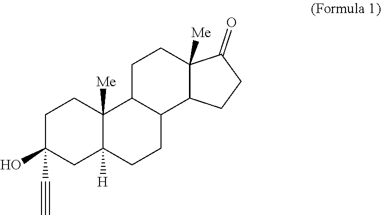 [SMILES: C#C[C@]1(O)CC[C@]2(C)C3CC[C@]4(C)C(=O)CCC4C3CC[C@H]2C1]

or a pharmaceutically acceptable salt, hydrate, prodrug or solvate thereof, for use as a medicament.

2. 3α-ethynyl-3β-hydroxy-5α-androstan-17-one as shown in Formula 1

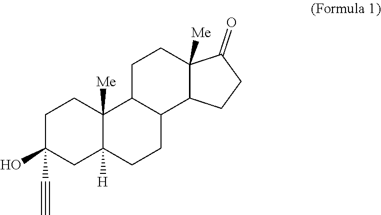 [SMILES: C#C[C@]1(O)CC[C@]2(C)C3CC[C@]4(C)C(=O)CCC4C3CC[C@H]2C1]

or a pharmaceutically acceptable salt, hydrate, prodrug or solvate thereof, for use in prevention, alleviation and/or treatment of a disease or disorder associated with an α3 subtype of the GABA_{A} receptor, such as the α3β2γ2 subtype of the GABA_{A} receptor.

3. The compound for use according to item 2, wherein said disease or disorder associated with an α3 subtype of the GABA_{A} receptor is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder, diabetes and autoimmune disease.

4. The compound for use according to item 2 or 3, wherein said disease or disorder is Prader-Willi's syndrome.

5. The compound for use according to item 2 or 3, wherein said disease or disorder is obesity associated with polycystic ovarian syndrome.

6. The compound for use according to item 2 or 3, wherein said disease or disorder is obesity associated with diabetes, such as obesity associated with type II diabetes.

7. The compound for use according to item 2 or 3, wherein said disease or disorder is obesity.

8. The compound for use according to item 2 or 3, wherein said disease or disorder is a hyperphagia disorder.

9. The compound for use according to any one of items 1 to 8, wherein said pharmaceutically acceptable salt is a sodium salt.

10. The compound for use according to any one of items 1 to 9, wherein use results in a decrease in bodyweight is seen after 1 to 20 days, such as after 3 to 15 days, such as after 5 to 10 days.

11. The compound for use according to any one of items 1 to 10, wherein said compound is administrated intravenously, subcutaneously, transdermally, nasally, per rectum, intravaginally, percutaneously, intramuscularly, or orally.

12. The compound for use according to any one of items 1 to 11, wherein said administration is oral or nasal administration.

13. The compound for use according to any one of items 1 to 12, wherein said compound is administrated in a dose in the range of from about 0.1 to about 300 mg per kg body weight, such as a dose in the range of from about 0.2 to about 200 mg per kg body weight, such as a dose in the range of from about 0.3 to about 150 mg, such as a dose in the range of from about 0.4 to about 150 mg per kg bodyweight, such as a dose in the range of from about 0.5 to about 120 mg per kg bodyweight, such as a dose in the range of from about 1 to about 100 mg per kg body weight, such as a dose in the range of from about 1 to about 50 mg per kg body weight, such as a dose in the range of from about 1 to about 5 mg per kg body weight, such as about 1 mg per kg body weight.

14. The compound for use according to any one of items 1 to 13, wherein said compound provides an antagonistic effect on the effect of γ-aminobutyric acid (GABA) and/or any GABA_{A} receptor modulating steroids (GAMS) on the α3 subtype of the GABA_{A} receptor.

15. The compound for use according to any one of items 1 to 14, wherein said compound provides an antagonistic effect on the effect of γ-aminobutyric acid (GABA) on the α3 subtype of the GABA_{A} receptor.

16. The compound for use according to any one of items 14 to 15, wherein said compound further provides an antagonistic effect on the effect of γ-aminobutyric acid (GABA) and/or any GABA_{A} receptor modulating steroids (GAMS) on the α2, α4 and/or α5 subtype(s) of the GABA_{A} receptor α2, α4 and/or α5 subtype(s).

17. The compound for use according to any one of items 14 to 16, wherein said compound further provides an antagonistic effect on the effect of γ-aminobutyric acid (GABA) on the α2, α4 and/or α5 subtype(s) of the GABA_{A} receptor α2, α4 and/or α5 subtype(s).

18. A method of treating, alleviating and/or preventing a disease or disorder associated with an α3 subtype of the GABA_{A} receptor, comprising administering a pharmaceutically effective amount of 3α-ethynyl-3β-hydroxy-5α-androstan-17-one as shown in Formula 1

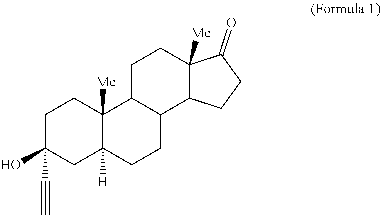 [SMILES: C#C[C@]1(O)CC[C@]2(C)C3CC[C@]4(C)C(=O)CCC4C3CC[C@H]2C1]

or a pharmaceutically acceptable salt, hydrate, prodrug or solvate thereof, to a patient in need thereof.

19. The method according to item 18, wherein said α3 subtype of the GABA_{A} receptor is the α3β2γ2 subtype of the GABA_{A} receptor.

20. The method according to any one of items 17 to 19, wherein said disease or disorder associated with an α3 subtype of the GABA_{A} receptor is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder, diabetes and autoimmune disease.

21. The method according to any one of items 18 to 20, wherein said obesity is associated with polycystic ovarian syndrome.

22. The method according to any one of items 18 to 20, wherein said obesity is associated with diabetes.

23. The method according to any one of items 18 to 20, wherein said disease or disorder is obesity.

24. The method according to any one of items 18 to 20, wherein said disease is an hyperphagia disorder.

25. The method according to any one of items 18 to 24, wherein said compound is in the form of a sodium salt.

26. The method according to any one of items 18 to 25, wherein method results in a decrease in bodyweight is seen after 1 to 20 days, such as after 3 to 15 days, such as after 5 to 10 days.

27. The method according to any one of items 18 to 26, wherein said 3α-ethynyl-3β-hydroxy-5α-androstan-17-one or a pharmaceutically acceptable salt, hydrate, prodrug or solvate thereof is administrated intravenously, subcutaneously, transdermally, nasally, per rectum, intravaginally, percutaneously, intramuscularly, or orally.

28. The method according to any one of items 18 to 27, wherein said 3α-ethynyl-3β-hydroxy-5α-androstan-17-one or a pharmaceutically acceptable salt, hydrate, prodrug or solvate thereof is administrated orally or nasally.

29. The method according to any one of items 18 to 28, wherein 3α-ethynyl-3β-hydroxy-5α-androstan-17-one or a pharmaceutically acceptable salt, hydrate, prodrug or solvate thereof is administrated in a dose in the range of from about 0.1 to about 300 mg per kg body weight, such as a dose in the range of from about 0.2 to about 200 mg per kg body weight, such as a dose in the range of from about 0.3 to about 150 mg, such as a dose in the range of from about 0.4 to about 150 mg per kg bodyweight, such as a dose in the range of from about 0.5 to about 120 mg per kg bodyweight, such as a dose in the range of from about 1 to about 100 mg per kg body weight, such as a dose in the range of from about 1 to about 50 mg per kg body weight, such as a dose in the range of from about 1 to about 5 mg per kg body weight, such as about 1 mg per kg body weight.

30. The method according to any one of items 18 to 29, wherein 3α-ethynyl-3β-hydroxy-5α-androstan-17-one or a pharmaceutically acceptable salt, hydrate, prodrug or solvate thereof provides an antagonistic effect on the effect of γ-aminobutyric acid (GABA) and/or any GABA_{A} receptor modulating steroids (GAMS) on the GABA_{A} receptor α3 subtype.

31. The method according to any one of items 18 to 30, wherein 3α-ethynyl-3β-hydroxy-5α-androstan-17-one or a pharmaceutically acceptable salt, hydrate, prodrug or solvate thereof provides an antagonistic effect on the effect of γ-aminobutyric acid (GABA) on the GABA_{A} receptor α3 subtype.

32. The method according to any one of items 18 to 31, wherein 3α-ethynyl-3β-hydroxy-5α-androstan-17-one or a pharmaceutically acceptable salt, hydrate, prodrug or solvate thereof further provides an antagonistic effect on the effect of γ-aminobutyric acid (GABA) and/or any GABA_{A} receptor modulating steroids (GAMS) on the GABA_{A} receptor α2, α4 and/or α5 subtype(s).

33. The method according to any one of items 18 to 32, wherein 3α-ethynyl-3β-hydroxy-5α-androstan-17-one or a pharmaceutically acceptable salt, hydrate, prodrug or solvate thereof further provides an antagonistic effect on the effect of γ-aminobutyric acid (GABA) on the GABA_{A} receptor α2, α4 and/or α5 subtype(s).

34. Use of 3α-ethynyl-3β-hydroxy-5α-androstan-17-one as shown in Formula 1 or a pharmaceutically acceptable salt, hydrate, prodrug or solvate thereof, for the manufacture of a medicament for the prevention, alleviation and/or treatment of a disease or disorder associated with an α3 subtype of the GABA_{A} receptor, such as the α3β2γ2 subtype of the GABA_{A} receptor

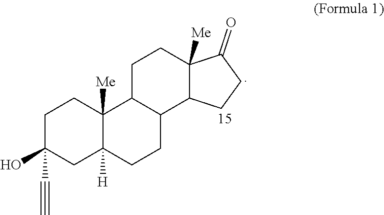 [SMILES: C#C[C@]1(O)CC[C@]2(C)C3CC[C@]4(C)C(=O)CCC4C3CC[C@H]2C1]

35. Use of 3α-ethynyl-3β-hydroxy-5α-androstan-17-one or a pharmaceutically acceptable salt, hydrate, prodrug or solvate thereof according to item 34, wherein said disease or disorder associated with an α3 subtype of the GABA_{A} receptor is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder, diabetes and autoimmune disease.

36. A pharmaceutical composition comprising 3α-ethynyl-3β-hydroxy-5α-androstan-17-one as shown in Formula 1 or a pharmaceutically acceptable salt, prodrug, hydrate or solvate thereof for use according to any one according to items 1 to 17, and at least one pharmaceutically acceptable excipient.

37. A pharmaceutical composition comprising 3α-ethynyl-3β-hydroxy-5α-androstan-17-one as shown in Formula 1 or a pharmaceutically acceptable salt, prodrug, hydrate or solvate thereof for use in a method according to any one according to items 18 to 33, and at least one pharmaceutically acceptable excipient.

38. A cosmetic composition comprising a cosmetically effective amount of 3α-ethynyl-3β-hydroxy-5α-androstan-17-one as shown in Formula 1

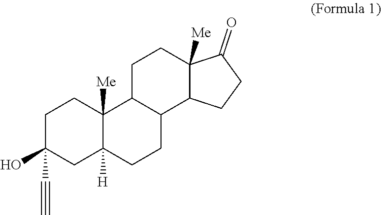 [SMILES: C#C[C@]1(O)CC[C@]2(C)C3CC[C@]4(C)C(=O)CCC4C3CC[C@H]2C1]

or a cosmetically acceptable salt, hydrate, precursor or solvate thereof and at least one cosmetically acceptable excipient.

39. Use of 3α-ethynyl-3β-hydroxy-5α-androstan-17-one as shown in Formula 1

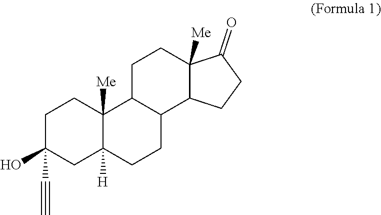 [SMILES: C#C[C@]1(O)CC[C@]2(C)C3CC[C@]4(C)C(=O)CCC4C3CC[C@H]2C1]

or a cosmetically acceptable salt, hydrate, precursor or solvate thereof or of a cosmetic composition according to item 38 for prevention and/or reduction of overweight.

40. Use of 3α-ethynyl-3β-hydroxy-5α-androstan-17-one as shown in Formula 1 or a cosmetically acceptable salt, hydrate, precursor or solvate thereof or of a cosmetic composition according to item 39, wherein said prevention or reduction of overweight is in a subject having a BMI<30.

41. Use of 3α-ethynyl-3β-hydroxy-5α-androstan-17-one as shown in Formula 1 or a cosmetically acceptable salt, hydrate, precursor or solvate thereof or of a cosmetic composition according to item 39 or 40, wherein overweight defined as a BMI in the range of 25-29.9.

42. Method of preventing or reducing overweight in a subject comprising administering a cosmetically effective amount of 3α-ethynyl-3β-hydroxy-5α-androstan-17-one as shown in Formula 1

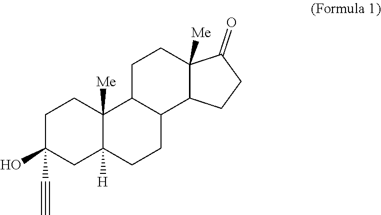 [SMILES: C#C[C@]1(O)CC[C@]2(C)C3CC[C@]4(C)C(=O)CCC4C3CC[C@H]2C1]

43. Method of preventing or reducing overweight according to item 42, wherein said prevention or reduction of overweight is in a subject having a BMI<30.

44. Method of preventing or reducing overweight according to item 42 or 43, wherein said overweight is defined as a BMI in the range of 25-29.9.

45. The use according to any one of items 39-41 or the method of preventing or reducing overweight according to any one of items 42 to 44, wherein a decrease in bodyweight is seen after 1 to 20 days, such as after 3 to 15 days, such as after 5 to 10 days.

1-15. (canceled)

16. A method of treating, alleviating and/or preventing a disease or disorder associated with an α3 subtype of a GABAA receptor, comprising administering a pharmaceutically effective amount of 3α-ethynyl-3β-hydroxy-5α-androstan-17-one as shown in Formula 1 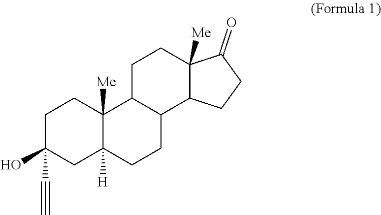 [SMILES: C#C[C@]1(O)CC[C@]2(C)C3CC[C@]4(C)C(=O)CCC4C3CC[C@H]2C1]
or a pharmaceutically acceptable salt, hydrate, prodrug or solvate thereof, to a patient in need thereof.

17. The method according to claim 16, wherein the α3 subtype of the GABAA receptor is the α3β2γ2 subtype of the GABAA receptor.

18. The method according to claim 16, wherein the disease or disorder associated with the α3 subtype of the GABAA receptor is selected from the group consisting of hyperphagia disorder; obesity; Prader-Willi's syndrome; polycystic ovarian syndrome; increased appetite disorder; obesity in diabetes; pathological food cravings; hypothalamic obesity; Cushing's syndrome; hyperphagia disorder associated with injury to the hypothalamus; alcoholism; substance use disorder; and relapses into alcohol and/or substance use disorder, diabetes and autoimmune disease.

19. The method according to claim 18, wherein the obesity is associated with polycystic ovarian syndrome.

20. The method according to claim 18, wherein the obesity is associated with diabetes.

21. The method according to claim 16, wherein the disease or disorder is obesity.

22. The method according to claim 16, wherein the disease is a hyperphagia disorder.

23. The method according to claim 16, wherein the compound is in the form of a sodium salt.

24. The method according to claim 16, wherein the method results in a decrease in bodyweight seen after 1 to 20 days.

25. The method according to claim 16, wherein the 3α-ethynyl-3β-hydroxy-5α-androstan-17-one or a pharmaceutically acceptable salt, hydrate, prodrug or solvate thereof is administrated intravenously, subcutaneously, transdermally, nasally, per rectum, intravaginally, percutaneously, intramuscularly, or orally.

26. The method according to claim 16, wherein the 3α-ethynyl-3β-hydroxy-5α-androstan-17-one or a pharmaceutically acceptable salt, hydrate, prodrug or solvate thereof is administrated orally or nasally.

27. The method according to claim 16, wherein 3α-ethynyl-3β-hydroxy-5α-androstan-17-one or a pharmaceutically acceptable salt, hydrate, prodrug or solvate thereof is administrated in a dose in the range of from about 0.1 to about 300 mg per kg body weight.

28. The method according to claim 16, wherein 3α-ethynyl-3β-hydroxy-5α-androstan-17-one or a pharmaceutically acceptable salt, hydrate, prodrug or solvate thereof provides an antagonistic effect on the effect of γ-aminobutyric acid (GABA) and/or any GABAA receptor modulating steroids (GAMS) on the GABAA receptor α3 subtype.

29. The method according to claim 16, wherein 3α-ethynyl-β3-hydroxy-5α-androstan-17-one or a pharmaceutically acceptable salt, hydrate, prodrug or solvate thereof provides an antagonistic effect on the effect of γ-aminobutyric acid (GABA) on the GABAA receptor α3 subtype.

30. The method according to claim 16, wherein 3α-ethynyl-3β-hydroxy-5α-androstan-17-one or a pharmaceutically acceptable salt, hydrate, prodrug or solvate thereof further provides an antagonistic effect on the effect of γ-aminobutyric acid (GABA) and/or any GABAA receptor modulating steroids (GAMS) on the GABAA receptor α2, α4 and/or α5 subtype(s).

31. The method according to claim 16, wherein 3α-ethynyl-3β-hydroxy-5α-androstan-17-one or a pharmaceutically acceptable salt, hydrate, prodrug or solvate thereof further provides an antagonistic effect on the effect of γ-aminobutyric acid (GABA) on the GABAA receptor α2, α4 and/or α5 subtype(s).

32-39. (canceled)

40. A non-therapeutic method of preventing or reducing overweight in a subject comprising administering a cosmetically effective amount of 3α-ethynyl-3β-hydroxy-5α-androstan-17-one as shown in Formula 1

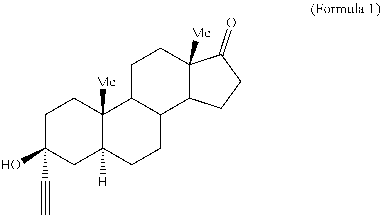 [SMILES: C#C[C@]1(O)CC[C@]2(C)C3CC[C@]4(C)C(=O)CCC4C3CC[C@H]2C1]

41. The non-therapeutic method of preventing or reducing overweight according to claim 40, wherein the prevention or reduction of overweight is in a subject having a BMI<30.

42. The non-therapeutic method of preventing or reducing overweight according to claim 40, wherein the overweight is defined as a BMI in the range of 25-29.9.

43. The non-therapeutic the method of preventing or reducing overweight according to claim 40, wherein a decrease in bodyweight is seen after 1 to 20 days.
